SEQUENCE LISTING (19 sequences)

SEQ ID NO: 1 - DNA, 21 bp, Artificial Sequence
  source: /note=“Description of Artificial Sequence Synthetic primer”
ccagtcgctatagctggcgta

SEQ ID NO: 2 - DNA, 58 bp, Artificial Sequence
  source: /note=“Description of Artificial Sequence Synthetic primer”
  source: /note=“Description of Combined DNA/RNA Molecule Synthetic primer”
aatgatacggcgaccaccgagatctacacctgacacaacactctttccctacacgaca

SEQ ID NO: 3 - DNA, 54 bp, Artificial Sequence
  source: /note=“Description of Artificial Sequence Synthetic primer”
  source: /note=“Description of Combined DNA/RNA Molecule Synthetic primer”
caagcagaagacggcatacgagataagcactggtgactggagttcagacgtgta

SEQ ID NO: 4 - RNA, 10 nt, Artificial Sequence
  source: /note=“Description of Artificial Sequence Synthetic oligonucleotide”
acaggaggca

SEQ ID NO: 5 - RNA, 10 nt, Artificial Sequence
  source: /note=“Description of Artificial Sequence Synthetic oligonucleotide”
uaaggaggug

SEQ ID NO: 6 - RNA, 19 nt, Artificial Sequence
  source: /note=“Description of Artificial Sequence Synthetic oligonucleotide”
gcucuuuaacaauuuauca

SEQ ID NO: 7 - RNA, 10 nt, Artificial Sequence
  source: /note=“Description of Artificial Sequence Synthetic oligonucleotide”
acauggauuc

SEQ ID NO: 8 - RNA, 10 nt, Artificial Sequence
  source: /note=“Description of Artificial Sequence Synthetic oligonucleotide”
uuaacuuuaa

SEQ ID NO: 9 - RNA, 10 nt, Artificial Sequence
  source: /note=“Description of Artificial Sequence Synthetic oligonucleotide”
uuaacgggaa

SEQ ID NO: 10 - RNA, 10 nt, Artificial Sequence
  source: /note=“Description of Artificial Sequence Synthetic oligonucleotide”
aaaaaaaaaa

SEQ ID NO: 11 - RNA, 15 nt, Artificial Sequence
  source: /note=“Description of Artificial Sequence Synthetic oligonucleotide”
uuaacuuuaaaaaaa

SEQ ID NO: 12 - RNA, 20 nt, Artificial Sequence
  source: /note=“Description of Artificial Sequence Synthetic oligonucleotide”
uuaacuuuaaaaaaaaaaaa

SEQ ID NO: 13 - RNA, 30 nt, Artificial Sequence
  source: /note=“Description of Artificial Sequence Synthetic oligonucleotide”
uuaacuuuaaaaaaaaaaaaaaaaaaaaaa

SEQ ID NO: 14 - RNA, 30 nt, Artificial Sequence
  source: /note=“Description of Artificial Sequence Synthetic oligonucleotide”
uuaacuuuaaacauggauucacauggauuc

SEQ ID NO: 15 - protein, 6 aa, Artificial Sequence
  source: /note=“Description of Artificial Sequence Synthetic 6xHis tag”
HHHHHH

SEQ ID NO: 16 - DNA, 29 bp, Artificial Sequence
  source: /note=“Description of Artificial Sequence Synthetic oligonucleotide”
aaaaaaaaaaaaaaaaaaaaaaaaaaacc

SEQ ID NO: 17 - DNA, 23 bp, Artificial Sequence
  source: /note=“Description of Artificial Sequence Synthetic oligonucleotide”
ttttttttttccagtcgctatag

SEQ ID NO: 18 - DNA, 25 bp, Artificial Sequence
  source: /note=“Description of Artificial Sequence Synthetic oligonucleotide”
ttttttttttttttttttttttttt

SEQ ID NO: 19 - DNA, 23 bp, Artificial Sequence
  source: /note=“Description of Artificial Sequence Synthetic oligonucleotide”
aaaaaaaaaaaaaaaaaaaaacc

(19) United States
(12) Patent Application Publication
(10) Pub. No.: US 2023/0203477 A1
(43) Pub. Date: Jun. 29, 2023

(54) NUCLEIC ACID ARTIFICIAL MINI-PROTEOME LIBRARIES

(71) Applicant: THE BROAD INSTITUTE, INC., Cambridge, MA (US)

(72) Inventor: Edward F. Fritsch, Concord, MA (US)

(21) Appl. No.: 17/927,112

(22) PCT Filed: May 26, 2021
(86) PCT No.: PCT/US21/34131

§ 371 Date: Nov. 22, 2022

Related U.S. Application Data
(60) Provisional application No. 63/030,056, filed on May 26, 2020.

(51) Int. Cl. C12N 15/10

(52) U.S. Cl. CPC C12N 15/1037; C12N 15/1062; C12N 15/1096

(57) ABSTRACT

Provided herein are nucleic acid artificial mini-proteome libraries, and methods of making and using such libraries.

### RELATED APPLICATIONS

This application is a §371 national-stage application based on PCT Application number PCT/US2021/034131, filed May 26, 2021, which claims the benefit of priority to U.S. Provisional Patent Application Ser. No. 63/030,056, filed May 26, 2020, each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 24, 2021, is named BRB-01525_(35500-01525)_SL.txt and is 5,320 bytes in size.

### BACKGROUND

The availability of nucleic acid artificial mini-proteome libraries enriched for sequences encoding open reading frames would have many different potential application applications. For example, such libraries would be valuable for the production of vaccines, and particularly cancer vaccines.

Vaccines have a long history in the treatment of cancers. Cancer vaccines are typically composed of tumor antigens and immunostimulatory molecules (e.g., cytokines or TLR ligands) that work together to activate antigen-specific cytotoxic T cells (CTLs) that recognize and lyse tumor cells. Such vaccines often contain either shared or patient-specific tumor antigens or whole tumor cell preparations. Shared tumor antigens are immunogenic proteins with selective expression in tumors across many individuals and are commonly delivered to patients as synthetic peptides, recombinant proteins, RNA or DNA vectors. Patient-specific tumor antigens that have been used in vaccines consists of proteins with tumor-specific mutations that result in altered amino acid sequences. Such mutated proteins have the potential to: (a) uniquely mark a tumor (relative to non-tumor cells) for recognition and destruction by the immune system; and (b) avoid central and sometimes peripheral T cell tolerance, and thus be recognized by more effective, high avidity T cells receptors. Whole tumor cell preparations contain all the potential antigens in a tumor cell and can be delivered to patients as autologous irradiated cells, cell lysates, cell fusions, heat-shock protein preparations or total mRNA (or cDNA/DNA vectors corresponding to total mRNA). When whole tumor cells are isolated from an autologous patient, the cells express patient-specific tumor antigens as well as shared tumor antigens.

Total mRNA from cells has been used to prepare cancer vaccines based on the total cell proteome. However, such mRNA samples can often be fragmented, particularly when it is obtained from a paraffin embedded (FFPE) sample. A problem with using fragmented mRNA from tumor cells as cancer vaccines is that most of the RNA fragments will not be in the proper reading frame for effective translation. Accordingly, there remains a need for improved nucleic acid mini-proteome libraries enriched for open reading frame fragments that are useful for producing cancer vaccines. In particular, there remains a need for preparation of improved nucleic acid mini-proteome libraries for preparation of personal vaccines based on the composition of the proteome in each individual.

### SUMMARY

Provided herein are compositions and methods related to the preparation of nucleic acid libraries enriched for sequences containing in-frame coding regions from fragmented RNA of a cell. Such libraries represent a mini-proteome of the cell, such that the nucleic acids in the library can be transferred into a suitable host cell to express the mini-proteome. In certain embodiments, such mini-proteome nucleic acid libraries are useful as tumor vaccines and/or in the preparation of tumor vaccines, particularly personal tumor vaccines prepared from the tumor RNA of an individual.

In certain aspects, provided herein are methods of enriching a library of in-frame coding region fragments from a population of RNA transcripts, or from a population of cellular RNA fragments (e.g., from a tumor). In some aspects, provided herein are methods of generating a tumor vaccine, or methods of treating a patient with a tumor using the generated tumor vaccine. In certain aspects, the present disclosure relates to libraries of purified polypeptide-linked RNA complexes, amplification products and vectors that comprise the enriched in-frame coding fragment sequences, tumor vaccines, and pharmaceutical compositions thereof.

In certain aspects, provided herein is a method of enriching a library of in-frame coding region fragments from a population of RNA transcripts, the method comprising: (a) generating a population of puromycin-tagged RNA transcripts, wherein: each RNA transcript in the population of puromycin-tagged RNA transcripts comprises, in 5′ to 3′ order: (i) a translation initiation site followed by any multiple of 3 nucleotides not encoding a stop codon; (ii) a RNA sequence transcribed from a cDNA fragment sequence from a library of cDNA sequences (e.g., from a tumor); (iii) a polypeptide-encoding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames; and wherein the 3′ end of each RNA transcript is joined to the 5′ end of a puromycin-tagged DNA linker; (b) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide-encoding nucleotide sequence, the puromycin will covalently link the translated polypeptide to the puromycin-tagged RNA transcript to form a polypeptide-linked RNA complex; and (c) separating the polypeptide-linked RNA complexes from the RNA transcripts that are not in such complexes, thereby enriching a library of in-frame coding region fragments from a population of RNA transcripts.

In certain embodiments, the population of puromycin-tagged RNA transcripts is generated by (a) contacting the RNA transcripts with splint polynucleotides and puromycin-tagged DNA linkers, wherein: the splint polynucleotides each comprise, in 3′ to 5′ order: (I) a sequence complementary to the 3′ end of the polypeptide-encoding nucleotide sequence; and (II) a poly-T sequence, the puromycin-tagged DNA linker each comprise, in 5′ to 3′ order: (1) a poly-dA sequence; and (2) a puromycin molecule, and wherein the polypeptide-encoding nucleotide sequence of the RNA transcripts hybridize to the sequence complementary to the 3′ end of the polypeptide-encoding nucleotide sequence of the splint polynucleotides, and the poly-dA sequence of the linker polynucleotides hybridize to the poly-T sequence of the splint polynucleotides; (b) performing a ligation reaction to ligate the 3′ end of the RNA transcripts to the 5′ end of the puromycin-tagged DNA linkers to generate puromycin-tagged RNA transcripts.

In certain aspects, provided herein is a method of enriching a library of in frame coding region fragments from a population of RNA transcripts, the method comprising: (a) generating a population of puromycin-tagged RNA transcripts, wherein: each RNA transcript in the library of puromycin-tagged RNA transcripts comprises, in 5′ to 3′ order: (i) a translation initiation site followed by any multiple of 3 nucleotides not encoding a stop codon; (ii) a polypeptide-encoding nucleotide sequence which is a multiple of 3 nucleotides in length and encoded by the reading frame initiating at the first 5′ nucleotide of the nucleotide sequence and lacks an in-frame stop codon in that reading frame; (iii) a RNA sequence transcribed from a cDNA fragment sequence from a library of cDNA sequences (e.g., from a tumor); and (iv) an adapter sequence which is a multiple of 3 nucleotides in length, and lacks stop codons in the reading frame beginning at the first 5′ nucleotide of the adapter sequence but contains stop codons in the other two reading frames, and wherein the 3′ end of each RNA transcript is joined to the 5′ end of a puromycin-tagged DNA linker; (b) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide-encoding nucleotide sequence, the puromycin will covalently link the translated polypeptide to the puromycin-tagged RNA transcript to form a polypeptide-linked RNA complex; and (c) separating the polypeptide-linked RNA complexes from the RNA transcripts that are not in such complexes, thereby enriching a library of in-frame coding region fragments from a population of RNA transcripts.

In certain embodiments, the population of puromycin-tagged RNA transcripts is generated by (a) contacting the RNA transcripts with splint polynucleotides and puromycin-tagged DNA linkers, wherein: the splint polynucleotides each comprise, in 3′ to 5′ order: (I) a sequence complementary to the adapter sequence; and (II)a poly-T sequence, the puromycin-tagged DNA linker each comprise, in 5′ to 3′ order: (1) a poly-dA sequence; and (2) a puromycin molecule, and wherein the polypeptide-encoding nucleotide sequence of the RNA transcripts hybridize to the sequence complementary to the adapter sequence of the splint polynucleotides, and the poly-dA sequence of the linker polynucleotides hybridize to the poly-T sequence of the splint polynucleotides; (b) performing a ligation reaction to ligate the 3′ end of the RNA transcripts to the 5′ end of the puromycin-tagged DNA linkers to generate puromycin-tagged RNA transcripts.

In some embodiments, the methods described herein further comprise the step of generating the library of RNA transcripts prior to step (a) by performing a transcription reaction on a library of RNA expression constructs, wherein each RNA expression construct comprises: (i) a transcription promoter; (ii) a translation initiation site followed by any multiple of 3 nucleotides not encoding a stop codon; (iii) a cDNA fragment sequence from a library of cDNA fragment sequences; and (iv) a polypeptide coding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames. In certain embodiments, the translation initiation site comprises a Shine-Dalgarno sequence.

In certain embodiments, each RNA expression construct further comprises an adapter sequence which is a multiple of 3 nucleotides in length, and lacks stop codons in the reading frame beginning at the first 5′ nucleotide of the adapter sequence but contains stop codons in the other two reading frames. In some embodiments, the library of cDNA fragment sequences is enriched for exome-containing cDNA fragments. In some embodiments, the library of cDNA fragment sequences is enriched for mismatch-containing cDNA fragment sequences.

In certain aspects, provided herein is a method of enriching a library of in-frame coding region fragments from a population of cellular RNA fragments (e.g., from a tumor), the method comprising: (a) performing strand-specific random primed nucleic acid amplification reaction on a population of cellular RNA fragments to generate a population of cDNA fragments; (b) contacting the population of cDNA fragments with exome capture probes thereby enriching the population of cDNA fragments for exome-encoding cDNA fragments to generate a library of exome-enriched cDNA fragments; (c) generating RNA expression constructs comprising, (i) a transcription promoter; (ii) a translation initiation site followed by any multiple of 3 nucleotides not encoding a stop codon; (iii) one of the exome-enriched cDNA fragments from the library of exome-enriched cDNA fragments; (iv) a polypeptide-coding nucleotide sequence which is a multiple of 3 nucleotides in length and encoded by the reading frame initiating at the first 5′ nucleotide of the nucleotide sequence and lacks an in-frame stop codon in that reading frame but contains stop codons in the other two reading frames; (d) performing a transcription reaction using the RNA expression constructs to generate a library of RNA transcripts each comprising, in 5′ to 3′ order: (i) a translation initiation site followed by any multiple of 3 nucleotides not encoding a stop codon; (ii) a RNA sequence transcribed from a cDNA fragment sequence of the library of exome-enriched cDNA fragments; (iii) a polypeptide-coding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames, (e) generating a population of puromycin-tagged RNA transcripts, wherein the 3′ end of each RNA transcript is joined to the 5′ end of a puromycin-tagged DNA linker; (f) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex; and (g) separating the polypeptide-linked RNA complexes from the RNA transcripts that are not in such complexes, thereby enriching a library of in-frame coding region fragments from a population of cellular RNA fragments. In certain embodiments, the translation initiation site comprises a Shine-Dalgarno sequence.

In certain embodiments, the population of puromycin-tagged RNA transcripts is generated by (a) contacting the RNA transcripts with splint polynucleotides and puromycin-tagged DNA linkers, wherein: the splint polynucleotides each comprise, in 3′ to 5′ order: (I) a sequence complementary to the 3′ end of the polypeptide-encoding nucleotide sequence; and (II) a poly-T sequence, the puromycin-tagged DNA linker each comprise, in 5′ to 3′ order: (1) a poly-dA sequence; and (2) a puromycin molecule, and wherein the polypeptide-encoding nucleotide sequence of the RNA transcripts hybridize to the sequence complementary to the 3′ end of the polypeptide-encoding nucleotide sequence of the splint polynucleotides, and the poly-dA sequence of the linker polynucleotides hybridize to the poly-T sequence of the splint polynucleotides; (b) performing a ligation reaction to ligate the 3′ end of the RNA transcripts to the 5′ end of the puromycin-tagged DNA linkers to generate puromycin-tagged RNA transcripts.

In certain aspects, provided herein is a method of enriching a library of in frame coding region fragments from a population of cellular RNA fragments (e.g., from a tumor), the method comprising: (a) performing strand-specific random primed nucleic acid amplification reaction on a population of cellular RNA fragments to generate a population of cDNA fragments; (b) contacting the population of cDNA fragments with exome capture probes thereby enriching the population of cDNA fragments for exome-encoding cDNA fragments to generate a library of exome-enriched cDNA fragments; (c) generating RNA expression constructs comprising, (i) a transcription promoter; (ii) a translation initiation site followed by any multiple of 3 nucleotides not encoding a stop codon; (iii) a polypeptide-coding nucleotide sequence which is a multiple of 3 nucleotides in length and encoded by the reading frame initiating at the first 5′ nucleotide of the nucleotide sequence and lacks an in-frame stop codon in that reading frame; (iv) one of the exome-enriched cDNA fragments from the library of exome-enriched cDNA fragments; and (v) an adapter sequence which is a multiple of 3 nucleotides in length, and contains no stop codons in the reading frame beginning at the first 5′ nucleotide of the adapter sequence and stop codons in each of the other reading frames; (d) performing a transcription reaction using the RNA expression constructs to generate a library of RNA transcripts each comprising, in 5′ to 3′ order: (i) a translation initiation site followed by any multiple of 3 nucleotides not encoding a stop codon; (ii) a polypeptide coding nucleotide sequence which is a multiple of 3 nucleotides in length and encoded by the reading frame initiating at the first 5′ nucleotide of the nucleotide sequence and lacks an in-frame stop codon in that reading frame; (iii) a RNA sequence transcribed from a cDNA fragment sequence of the library of exome-enriched cDNA fragments; and (iv) an adapter sequence which is a multiple of 3 nucleotides in length, and contains no stop codons in the reading frame beginning at the first 5′ nucleotide of the adapter sequence and stop codons in each of the other reading frames, (e) generating a population of puromycin-tagged RNA transcripts, wherein the 3′ end of each RNA transcript is joined to the 5′ end of a puromycin-tagged DNA linker; (f) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex; and (g) separating the polypeptide-linked RNA complexes from the RNA transcripts that are not in such complexes, thereby enriching a library of in-frame coding region fragments from a population of cellular RNA fragments. In certain embodiments, the translation initiation site comprises a Shine-Dalgarno sequence.

In certain embodiments, the population of puromycin-tagged RNA transcripts is generated by (a) contacting the RNA transcripts with splint polynucleotides and puromycin-tagged DNA linkers, wherein: the splint polynucleotides each comprise, in 3′ to 5′ order: (I) a sequence complementary to the adapter sequence; and (II) a poly-T sequence, the puromycin-tagged DNA linker each comprise, in 5′ to 3′ order: (1) a poly-dA sequence; and (2) a puromycin molecule, and wherein the polypeptide-encoding nucleotide sequence of the RNA transcripts hybridize to the sequence complementary to the adapter sequence of the splint polynucleotides, and the poly-dA sequence of the linker polynucleotides hybridize to the poly-T sequence of the splint polynucleotides; (b) performing a ligation reaction to ligate the 3′ end of the RNA transcripts to the 5′ end of the puromycin-tagged DNA linkers to generate puromycin-tagged RNA transcripts.

In some embodiments, step (b) of the methods of enriching a library of in frame coding region fragments from a population of cellular RNA fragments described herein further comprises contacting the population of cDNA fragments with a MutS protein, thereby enriching the population of cDNA fragments for mismatch-containing cDNA fragments due to either mutations or to single nucleotide polymorphisms. In some embodiments, step (b) of the methods of enriching a library of in frame coding region fragments from a population of cellular RNA fragments described herein further comprises contacting the library of exome-enriched cDNA fragments with a MutS protein, thereby enriching the library of exome-enriched cDNA fragments for mismatch-containing cDNA fragments due to either mutations or to single nucleotide polymorphisms.

In some embodiments, the methods of enriching a library of in frame coding region fragments from a population of cellular RNA fragments described herein further comprise the step of preparing the population of cellular RNA fragments from a sample. In some embodiments, the sample is a tumor sample, a normal tissue sample, a diseased tissue sample, a fresh sample, a frozen sample, and/or a paraffin embedded (FFPE) sample. In certain embodiments, the sample is a paraffin embedded (FFPE) tissue or tumor sample. In some embodiments, the methods of enriching a library of in frame coding region fragments from a population of cellular RNA fragments described herein further comprise obtaining the sample from a subject (e.g., a cancer patient). In some embodiments, the cellular RNA fragments in the population of cellular RNA fragments are of between 150 and 250 nt in length (e.g., about 200 nt in length).

In some embodiments, the polypeptide-linked RNA complexes is separated from the RNA transcripts that are not in such complexes by affinity purifying the polypeptide-linked RNA complexes using a reagent that binds to the polypeptide encoded by the polypeptide-encoding nucleotide sequence. In some embodiments, the methods described herein further comprise performing an RT-PCR amplification reaction on the purified protein-linked RNA complexes to generate an amplification product comprising an amplified DNA copy of the cDNA fragment sequence. In some embodiments, the methods described herein further comprise inserting the amplification product into a vector (e.g., a cloning vector, an expression vector, or a vaccine-coding vector) to generate vectors comprising the sequence of the cDNA fragments. In certain embodiments, the methods described herein further comprise contacting the amplification products with a MutS protein, thereby enriching the amplification products for mismatch-containing cDNA fragments due to either mutations or to single nucleotide polymorphisms.

In some embodiments, the methods described herein further comprise inserting the vaccine-coding vectors into bacteria and incubating the bacteria under conditions such that they express the vaccine encoded by the vaccine-coding vector. In some embodiments, the methods described herein further comprise the vaccine-coding vectors into yeast and incubating the yeast under conditions such that they express the vaccine encoded by the vaccine-coding vector. In some embodiments, the methods described herein further comprise subjecting the vaccine-coding vectors to an in vitro translation reaction to generate the vaccine encoded by the vaccine-coding vector. In some embodiments, the methods described herein further comprise transfecting or transducing the vectors into mammalian cells (e.g., human cells) and incubating the mammalian cells under conditions such that they express the vaccine encoded by the vector. In some embodiments, the methods described herein further comprise transfecting or transducing the vectors into mammalian cells (e.g., human cells) ex vivo and delivering the mammalian cells to a subject (e.g., a human, and preferably a cancer patient). In certain embodiments, the mammalian cells (e.g., human cells) are primary T cells or antigen-presenting cells isolated from the same subject or a different subject. In some embodiments, the methods described herein further comprise delivering the vectors to a subject (e.g., a human, and preferably a cancer patient) such that the subject expresses the vaccine encoded by the vector.

In certain aspects, provided herein is a library of purified polypeptide-linked RNA complexes generated according to the methods described herein.

In certain aspects, provided herein are amplification products generated according to methods described herein.

In certain aspects, provided herein are vectors (e.g., cloning vectors, expression vectors, or vaccine-coding vectors) generated according to methods described herein.

In certain aspects, provided herein is a pharmaceutical composition comprising an amplification product generated according to methods described herein and a pharmaceutically acceptable carrier.

In certain aspects, provided herein is a pharmaceutical composition comprising a vector generated according to methods described herein and a pharmaceutically acceptable carrier.

In certain aspects, provided herein is a method of generating a tumor vaccine comprising:

(a) generating cellular RNA fragments from a tumor sample of a subject; (b) performing strand-specific random primed nucleic acid amplification reaction on the RNA fragments to generate cDNA fragments; (c) contacting the cDNA fragments with exome capture probes thereby enriching the cDNA fragments for exome-encoding cDNA fragments to generate a library of exome-enriched cDNA fragments; (d) generating RNA expression constructs comprising, (i) a transcription promoter; (ii) a translation initiation site followed by any multiple of 3 nucleotides not encoding a stop codon; (iii) one of the exome-enriched cDNA fragments from the library of exome-enriched cDNA fragments; (iv) a polypeptide coding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames; (e) performing a transcription reaction using the RNA expression constructs to generate a library of RNA transcripts each comprising, in 5′ to 3′ order: (i) a translation initiation site followed by any multiple of 3 nucleotides not encoding a stop codon; (ii) a RNA sequence transcribed from a cDNA fragment sequence of the library of exome-enriched cDNA fragments; (iii) a polypeptide-coding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames, (f) generating a population of puromycin-tagged RNA transcripts, wherein the 3′ end of each RNA transcript is joined to the 5′ end of a puromycin-tagged DNA linker; (g) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex; (h) affinity purifying the polypeptide-linked RNA complexes using a reagent that binds to the polypeptide encoded by the polypeptide coding nucleotide sequence to generate a library of purified polypeptide-linked RNA complexes; (i) performing an amplification reaction on the library of purified polypeptide-linked RNA complexes to generate amplification products comprising the sequence of the cDNA fragments; and (j) generating a tumor vaccine from one or more of the amplification products of step (i). In certain embodiments, the translation initiation site comprises a Shine-Dalgarno sequence.

In certain embodiments, the population of puromycin-tagged RNA transcripts is generated by (a) contacting the RNA transcripts with splint polynucleotides and puromycin-tagged DNA linkers, wherein: the splint polynucleotides each comprise, in 3′ to 5′ order: (I) a sequence complementary to the 3′ end of the polypeptide-encoding nucleotide sequence; and (II) a poly-T sequence, the puromycin-tagged DNA linker each comprise, in 5′ to 3′ order: (1) a poly-dA sequence; and (2) a puromycin molecule, and wherein the polypeptide-encoding nucleotide sequence of the RNA transcripts hybridize to the sequence complementary to the 3′ end of the polypeptide-encoding nucleotide sequence of the splint polynucleotides, and the poly-dA sequence of the linker polynucleotides hybridize to the poly-T sequence of the splint polynucleotides; (b) performing a ligation reaction to ligate the 3′ end of the RNA transcripts to the 5′ end of the puromycin-tagged DNA linkers to generate puromycin-tagged RNA transcripts.

In certain aspects, provided herein is a method of generating a tumor vaccine comprising: (a) generating cellular RNA fragments from a tumor sample of a subject; (b) performing strand-specific random primed nucleic acid amplification reaction on the cellular RNA fragments to generate cDNA fragments; (c) contacting the cDNA fragments with exome capture probes thereby enriching the cDNA fragments for exome-encoding cDNA fragments to generate a library of exome-enriched cDNA fragments; (d) generating RNA expression constructs comprising, (i) a transcription promoter; (ii) a translation initiation site followed by any multiple of 3 nucleotides not encoding a stop codon; (iii) a polypeptide coding nucleotide sequence which is a multiple of 3 nucleotides in length and encoded by the reading frame initiating at the first 5′ nucleotide of the nucleotide sequence and lacks an in-frame stop codon in that reading frame; (iv) one of the exome-enriched cDNA fragments from the library of exome-enriched cDNA fragments; and (v) an adapter sequence which is a multiple of 3 nucleotides in length, and contains no stop codons in the reading frame beginning at the first 5′ nucleotide of the adapter sequence and stop codons in each of the other reading frames; (e) performing a transcription reaction using the RNA expression constructs to generate a library of RNA transcripts each comprising, in 5′ to 3′ order: (i) a translation initiation site followed by any multiple of 3 nucleotides not encoding a stop codon; (ii) a polypeptide coding nucleotide sequence which is a multiple of 3 nucleotides in length and encoded by the reading frame initiating at the first 5′ nucleotide of the nucleotide sequence and lacks an in-frame stop codon in that reading frame; (iii) a RNA sequence transcribed from a cDNA fragment sequence of the library of exome-enriched cDNA fragments; and (iv) an adapter sequence which is a multiple of 3 nucleotides in length, and contains no stop codons in the reading frame beginning at the first 5′ nucleotide of the adapter sequence and stop codons in each of the other reading frames, (f) generating a population of puromycin-tagged RNA transcripts, wherein the 3′ end of each RNA transcript is joined to the 5′ end of a puromycin-tagged DNA linker; (g) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex; (h) affinity purifying the polypeptide-linked RNA complexes using a reagent that binds to the polypeptide encoded by the polypeptide coding nucleotide sequence to generate a library of purified polypeptide-linked RNA complexes; (i) performing an amplification reaction on the library of purified polypeptide-linked RNA complexes to generate amplification products comprising the sequence of the cDNA fragments; and (j) generating a tumor vaccine from one or more of the amplification products of step (i). In certain embodiments, the translation initiation site comprises a Shine-Dalgarno sequence.

In certain embodiments, the population of puromycin-tagged RNA transcripts is generated by (a) contacting the RNA transcripts with splint polynucleotides and puromycin-tagged DNA linkers, wherein: the splint polynucleotides each comprise, in 3′ to 5′ order: (I) a sequence complementary to the adapter sequence; and (II) a poly-T sequence, the puromycin-tagged DNA linker each comprise, in 5′ to 3′ order: (1) a poly-dA sequence; and (2) a puromycin molecule, and wherein the polypeptide-encoding nucleotide sequence of the RNA transcripts hybridize to the sequence complementary to the adapter sequence of the splint polynucleotides, and the poly-dA sequence of the linker polynucleotides hybridize to the poly-T sequence of the splint polynucleotides; (b) performing a ligation reaction to ligate the 3′ end of the RNA transcripts to the 5′ end of the puromycin-tagged DNA linkers to generate puromycin-tagged RNA transcripts.

In some embodiments, the sample is a tumor sample, a normal tissue sample, a diseased tissue sample, a fresh sample, a frozen sample, and/or a paraffin embedded (FFPE) sample. In certain embodiments, the sample is a paraffin embedded (FFPE) tissue or tumor sample. In some embodiments, the methods of generating a tumor vaccine described herein further comprise obtaining the sample from a subject (e.g., a cancer patient). In some embodiments, the cellular RNA fragments in the population of cellular RNA fragments are of between 150 and 250 nt in length (e.g., about 200 nt in length).

In some embodiments, the methods of generating a tumor vaccine described herein further comprise inserting the amplification product into a vaccine-coding vector to generate vaccine-coding vectors comprising the sequence of the cDNA fragments prior to step (j).

In some embodiments, step (j) of the methods of generating a tumor vaccine comprises inserting the vaccine-coding vectors into bacteria and incubating the bacteria under conditions such that they express the vaccine encoded by the vaccine-coding vector.

In some embodiments, step (j) of the methods of generating a tumor vaccine comprises inserting the vaccine-coding vectors into yeast and incubating the yeast under conditions such that they express the vaccine encoded by the vaccine-coding vector.

In some embodiments, step (j) of the methods of generating a tumor vaccine comprises subjecting the vaccine-coding vectors to an in vitro translation reaction to generate the vaccine encoded by the vaccine-coding vector.

In some embodiments, step (j) of the methods of generating a tumor vaccine comprises inserting the vaccine-coding vectors into mammalian cells (e.g., human cells) and incubating the mammalian cells under conditions such that they express the vaccine encoded by the vaccine-coding vector.

In some embodiments, step (j) of the methods of generating a tumor vaccine comprises delivering the vaccine-coding vectors to a subject (e.g., a human and preferably a cancer patient) such that the subject expresses the vaccine encoded by the vaccine-coding vector.

In some embodiments, step (j) of the methods of generating a tumor vaccine comprises transfecting or transducing the vaccine-coding vectors into human cells ex vivo and delivering the human cells to a subject. In certain embodiments, the human cells are primary T cells or antigen-presenting cells isolated from the same subject or a different subject.

In some embodiments, the methods of generating a tumor vaccine described herein further comprise administering the tumor vaccine or cells containing the tumor vaccine to a subject (e.g., a human and preferably a cancer patient).

In certain aspect, provided herein is a method of treating a tumor, the method comprising administering the tumor vaccine generated according to methods described herein to a subject (e.g., a human and preferably a cancer patient) in need thereof.

In certain aspect, provided herein is a method of identifying drug targets comprising transfecting or transducing vectors generated according to methods described herein to cells and identifying in-frame coding region fragments that lead to a selectable phenotype. In some embodiments, the vectors are transfected or transduced to cells in vitro or in vivo. In certain embodiments, the in-frame coding region fragments are either enriched or depleted in the cells with the selectable phenotype. In certain embodiments, the in-frame coding region fragments positively or negatively alter an intracellular pathway. In certain embodiments, the cells are normal cells and the selectable phenotype is a disease phenotype.

In certain aspects, provided herein is a method of enriching a library of in-frame coding region fragments from a population of cellular RNA fragments, the method comprising: (a) performing strand-specific random primed nucleic acid amplification reaction on a population of cellular RNA fragments to generate a population of cDNA fragments; (b) inserting the population of cDNA fragments into cloning vectors to generate a library of DNA constructs, wherein each DNA construct comprises, in 5′ to 3′ order: (i) a promoter; (ii) a translation initiation site followed by any multiple of 3 nucleotides not encoding a stop codon; (iii) a polypeptide-encoding nucleotide sequence which is a multiple of 3 nucleotides in length and encoded by the reading frame initiating at the first 5′ nucleotide of the nucleotide sequence and lacks an in-frame stop codon in that reading frame; (iv) a cDNA fragment from the population of cDNA fragments; and (v) a membrane-presenting protein-encoding sequence, (c) transforming the library of DNA constructs into cells, (d) incubating the cells under conditions such that they express the DNA constructs; (e) purifying the cells (e.g., affinity purifying the cells) that express a complete fusion protein comprising the polypeptide encoded by the polypeptide-encoding nucleotide sequence, the polypeptide encoded by the cDNA fragment, and the membrane-presenting protein using a reagent that binds to the polypeptide encoded by the polypeptide-encoding nucleotide sequence; (f) recovering in-frame cDNA fragment sequences from the purified cells (e.g., by PCR amplification), thereby enriching a library of in-frame coding region fragments from a population of cellular RNA fragments.

In certain aspects, provided herein is a method of generating a tumor vaccine comprising: (a) generating cellular RNA fragments from a tumor sample of a subject; (b) performing strand-specific random primed nucleic acid amplification reaction on the RNA fragments to generate cDNA fragments; (c) inserting the population of cDNA fragments into cloning vectors to generate a library of DNA constructs, wherein each DNA construct comprises, in 5′ to 3′ order: (i) a promoter; (ii) a translation initiation site followed by any multiple of 3 nucleotides not encoding a stop codon; (iii) a polypeptide-encoding nucleotide sequence which is a multiple of 3 nucleotides in length and encoded by the reading frame initiating at the first 5′ nucleotide of the nucleotide sequence and lacks an in-frame stop codon in that reading frame; (iv) one cDNA fragment from the population of cDNA fragments; and (v) a membrane-presenting protein-encoding sequence, (d) transforming the library of DNA constructs into cells, (e) incubating the cells under conditions such that they express the DNA constructs; (f) purifying (e.g., affinity purifying) the cells that express a complete fusion protein comprising the polypeptide encoded by the polypeptide-encoding nucleotide sequence, the polypeptide encoded by the cDNA fragment, and the membrane-presenting protein using a reagent that binds to the polypeptide encoded by the polypeptide-encoding nucleotide sequence; (g) recovering in-frame cDNA fragment sequences from the purified cells (e.g., by PCR amplification), (h) generating a tumor vaccine from one or more of the amplification products of step (g).

### BRIEF DESCRIPTION OF FIGURES

FIG. 1 is a schematic diagram showing synthesis of stranded double-stranded (ds) cDNA. If desired, to capture open reading frames (ORFs) from anti-sense RNA, the identical library can be used but an opposite sense exome capture mix is required and the strand specificity of the primers for subsequent steps is reversed.

FIG. 2 is a schematic diagram showing MutS enrichment (optional) and Exome capture.

FIG. 3 is a schematic diagram showing preparation of RNA for display. The small protein coding sequence can be added to the 5′ upstream or 3′ downstream region of the cDNA fragment sequence from a cDNA library.

FIG. 4 is a schematic diagram showing RNA display.

FIG. 5 is a schematic diagram showing capture and recovery of polypeptide-linked RNA (AMPL-NA library fragments).

FIG. 6 is a schematic diagram showing an exemplary cloning process for membrane surface display.

FIG. 7 is a schematic diagram showing transformation, growth and surface presentation of in-frame library members according to certain exemplary embodiments disclosed herein.

FIG. 8 is a schematic diagram showing affinity enrichment of in-frame library and DNA recovery according to certain exemplary embodiments disclosed herein.

FIG. 9 is a schematic diagram showing the structure of an exemplary exome capture transcription library. RBS is an E. coli ribosome binding site, ATG is the initiation codon for protein translation, Readl and Read2 are Illumina TruSeq sequences, Twin-Strep-tag is the coding sequence for a 28-amino acid peptide used for binding purification, and Peptide is the coding sequence for a peptide spacer segment.

FIG. 10 shows results comparing full-length inserts with intact ORFs in the target reading frame for the constructs after Exome capture (“Before RNA Display”) and following RNA Display (“After RNA Display”).

### DETAILED DESCRIPTION

### General

In certain aspects, provided herein are methods of enriching a library of in-frame coding region fragments from a population of RNA transcripts, or from a population of cellular RNA fragments. In some aspects, provided herein are methods of generating a tumor vaccine, or methods of treating a patient with a tumor using the generated tumor vaccine. In certain aspects, the present disclosure relates to libraries of purified polypeptide-linked RNA complexes, amplification products and vectors that comprise the enriched in-frame coding fragment sequences, tumor vaccines, and pharmaceutical compositions thereof.

In certain aspects, the present disclosure relates to methods of preparing a nucleic acid library from fragmented RNA of a cell containing the proper in frame coding regions to represent a mini-proteome of the cell (a mini-proteome is defined here as a collection of ˜70 amino acid segments representing the expressed RNA coding potential of a cell), such that the nucleic acid can be transferred into a suitable host cell to express the mini-proteome.

There have been many challenges associated with preparation of such a library. The difficulties to prepare such a library are (a) that because an exogenous translation initiation site is required, there is no way to control that randomly fragmented RNA will be translated in the natural reading frame that encodes the native protein and (b) to control that it will exit the fragmented RNA in a reading frame that does not quickly terminate and hence be rapidly degraded by nonsense-mediated decay once inserted into a suitable host cell. Thus, without the solution provided herein, nearly 90% of the library members will be non-representative or not functional.

The methods provided by the present disclosure allow the enrichment out of a complex mixture of ˜200 nt RNA fragments from a cell those fragments which will be successfully translated in frame and enter the downstream region in the desired reading frame, thus eliminating 89% of RNAs that are not suitable for construction of a mini-proteome library.

Such a library is useful for preparation of a nucleic acid anti-tumor vaccine if the RNA is derived from a tumor cell or for identification of portions of the mini-proteome which alter, positively or negatively, intracellular (in vitro, i.e., in cell culture, or in vivo) pathways leading to selectable phenotypes that can identify new or more highly refined targets for pharmaceutical product discovery.

### Definitions

For convenience, certain terms employed in the specification, examples, and appended claims are collected here.

The articles “a” and “an” are used herein to refer to one or to more than one (e.g., to at least one) of the grammatical object of the article. By way of example, “an element” means one element or more than one element.

The term “barcoded primer” refers to a primer comprising a unique nucleotide sequence. The minimal length of this nucleotide sequence depends on the total number of primers that need to be uniquely labeled. For example, a nucleotide sequence that is 4 nucleotides long can have 256 different sequences, which can uniquely label up to 256 primers. The term “barcode-labeled amplification product is generated with these “barcoded primer” by PCR amplification reaction.

The term “binding” or “interacting” refers to an association, which may be a stable association, between two molecules, e.g., between an antibody and target, e.g., due to, for example, electrostatic, hydrophobic, ionic and/or hydrogen-bond interactions under physiological conditions.

As used herein, two nucleic acid sequences “complement” one another or are “complementary” to one another if they base pair one another at each position or at a fraction of all the positions.

As used herein, two nucleic acid sequences “correspond” to one another if they are both complementary to the same nucleic acid sequence.

The term “modulation” or “modulate”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity, or process. In certain instances, such regulation may be contingent on the occurrence of a specific event, such as activation of a signal transduction pathway, and/or may be manifest only in particular cell types.

The terms “polynucleotide” and “nucleic acid” are used herein interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

The term “neoantigen” or “neoantigenic” means a class of tumor antigens that arises from a tumor-specific mutation(s) which alters the amino acid sequence of genome encoded proteins.

A “vaccine” is to be understood as meaning a composition for generating immunity for the prophylaxis and/or treatment of diseases (e.g., tumor). Accordingly, vaccines are medicaments which comprise antigens and are intended to be used in humans or animals for generating specific defense and protective substance by vaccination.

As used herein, the term “administering” means providing a pharmaceutical agent or composition to a subject, and includes, but is not limited to, administering by a medical professional and self-administering.

As used herein, the term “subject” means a human or non-human animal selected for treatment or therapy.

Unless the context clearly indicates otherwise, “protein,” “polypeptide,” and “peptide” are used interchangeably herein when referring to a gene expression product, e.g., an amino acid sequence as encoded by a coding sequence. A “protein” may also refer to an association of one or more proteins, such as an antibody. A “protein” may also refer to a protein fragment. A protein may be a post-translationally modified protein such as a glycosylated protein. By “gene expression product” is meant a molecule that is produced as a result of transcription of an entire or part of a gene. Gene products include RNA molecules transcribed from a gene, as well as proteins translated from such transcripts. Proteins may be naturally occurring isolated proteins or may be the product of recombinant or chemical synthesis. The term “protein fragment” refers to a protein in which amino acid residues are deleted as compared to the reference protein itself, but where the remaining amino acid sequence is usually identical to at least a portion of that of the reference protein. Such deletions may occur at the amino-terminus or carboxy-terminus of the reference protein or at some internal position of the reference protein, or at more than one such position. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. Fragments of may be obtained using proteinases to fragment a larger protein, or by recombinant methods, such as the expression of only part of a protein-encoding nucleotide sequence (either alone or fused with another protein-encoding nucleic acid sequence). In various embodiments, a fragment may comprise an enzymatic activity and/or an interaction site of the reference protein to, e.g., a cell receptor. In another embodiment, a fragment may have immunogenic properties. The proteins may include mutations introduced at particular loci by a variety of known techniques, which do not adversely effect, but may enhance, their use in the methods provided herein. A fragment can retain one or more of the biological activities of the reference protein.

“Vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the invention is intended to include such other forms of expression vectors which serve equivalent functions and which become subsequently known in the art.

Unless otherwise defined herein, scientific and technical terms used in this application shall have the meanings that are commonly understood by those of ordinary skill in the art. Generally, nomenclature and techniques relating to chemistry, molecular biology, cell and cancer biology, immunology, microbiology, pharmacology, and protein and nucleic acid chemistry, described herein, are those well-known and commonly used in the art.

### Methods of Enriching a Library of in Frame Coding Region Fragments

In certain aspects, provided herein are methods of enriching a library of in-frame coding region fragments from a population of RNA transcripts. In certain embodiments, such methods comprise (a) generating a population of puromycin-tagged RNA transcripts; (b) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in-frame with the polypeptide-encoding nucleotide sequence, the puromycin will covalently link the translated polypeptide to the puromycin-tagged RNA transcript to form a polypeptide-linked RNA complex; and (c) separating the polypeptide-linked RNA complexes from the RNA transcripts that are not in such complexes, thereby enriching a library of in-frame coding region fragments from a population of RNA transcripts.

In some embodiments, each RNA transcript in the population of RNA transcripts comprises, in 5′ to 3′ order: (i) a translation initiation site; (ii) a RNA sequence transcribed from a cDNA fragment sequence from a library of cDNA sequences; (iii) a polypeptide-encoding nucleotide sequence which lacks an in-frame stop codon in the reading frame initiating at the first 5′ nucleotide of the nucleotide sequence but contains stop codons in each of the other two reading frames.

In certain embodiments, the population of puromycin-tagged RNA transcripts is generated by (a) contacting the RNA transcripts with splint polynucleotides and puromycin-tagged DNA linkers, wherein: the splint polynucleotides each comprise, in 3′ to 5′ order: (I) a sequence complementary to the 3′ end of the polypeptide-encoding nucleotide sequence; and (II) a poly-T sequence, the puromycin-tagged DNA linker each comprise, in 5′ to 3′ order: (1) a poly-dA sequence; and (2) a puromycin molecule, and wherein the polypeptide-encoding nucleotide sequence of the RNA transcripts hybridize to the sequence complementary to the 3′ end of the polypeptide-encoding nucleotide sequence of the splint polynucleotides, and the poly-dA sequence of the linker polynucleotides hybridize to the poly-T sequence of the splint polynucleotides; (b) performing a ligation reaction to ligate the 3′ end of the RNA transcripts to the 5′ end of the puromycin-tagged DNA linkers to generate puromycin-tagged RNA transcripts.

In some embodiments, each RNA transcript in the library of RNA transcripts comprises, in 5′ to 3′ order: (i) a translation initiation site; (ii) a polypeptide-encoding nucleotide sequence which lacks an in-frame stop codon in the reading frame initiating at the first 5′ nucleotide of the nucleotide sequence; (iii) a RNA sequence transcribed from a cDNA fragment sequence from a library of cDNA sequences; and (iv) an adapter sequence which lacks stop codons in the reading frame beginning at the first 5′ nucleotide of the adapter sequence but contains stop codons in the other two reading frames.

In certain embodiments, the population of puromycin-tagged RNA transcripts is generated by (a) contacting the RNA transcripts with splint polynucleotides and puromycin-tagged DNA linkers, wherein: the splint polynucleotides each comprise, in 3′ to 5′ order: (I) a sequence complementary to the adapter sequence; and (II)a poly-T sequence, the puromycin-tagged DNA linker each comprise, in 5′ to 3′ order: (1) a poly-dA sequence; and (2) a puromycin molecule, and wherein the polypeptide-encoding nucleotide sequence of the RNA transcripts hybridize to the sequence complementary to the adapter sequence of the splint polynucleotides, and the poly-dA sequence of the linker polynucleotides hybridize to the poly-T sequence of the splint polynucleotides; (b) performing a ligation reaction to ligate the 3′ end of the RNA transcripts to the 5′ end of the puromycin-tagged DNA linkers to generate puromycin-tagged RNA transcripts.

The translation start site of the RNA transcript may comprise a start codon (e.g., AUG), a Shine-Dalgarno (SD) sequence and/or translational enhancers. The Shine-Dalgarno sequence is a ribosomal binding site that commonly presents in bacterial and archaeal messenger RNA and generally located around 8 bases upstream of the start codon AUG. The Shine-Dalgarno sequence may comprise AGGAGG, AGGAGGU, GAGG, ACAGGAGGCA (SEQ ID NO: 4), or UAAGGAGGUG (SEQ ID NO: 5). The translational enhancer may comprise an A/U-rich enhancer, for example, 5′-GCUCUUUAACAAUUUAUCA-3′ (SEQ ID NO: 6), 5′-ACAUGGAUUC-3′ (SEQ ID NO: 7), 5′-UUAACUUUAA-3′ (SEQ ID NO: 8), 5′-UUAACGGGAA-3′ (SEQ ID NO: 9), 5′-AAAAAAAAAA-3′ (SEQ ID NO: 10), 5′-UUAACUUUAA-(A)₅-3′ (SEQ ID NO: 11), 5′-UUAACUUUAA-(A)₁₀-3′ (SEQ ID NO: 12), 5′-UUAACUUUAA-(A)₂₀-3′ (SEQ ID NO: 13), or 5′-UUAACUUUAA-(ACAUGGAUUC)₂-3′ (SEQ ID NO: 14). The translation start site may comprise a short (10-20 nucleotide) stretch of A residues between the translation enhancer sequence and the Shine-Dalgarno sequence to further improve translation efficiency.

In some embodiments, the translation initiation site of the RNA transcript is followed by any multiple of 3 nucleotides not encoding a stop codon. For example, the translation initiation site may be followed by 0, 3, 6, 9, 12, 15, 18, 21, 24, 27, 30, 33, 36, 39, 42, 45, 48, 51, 54, 57, 60, 63, 66, 69, 72, 75, 90, 120, 150, 180, 210, 240, 270, 300, 450, 600, 750, 900, 1200, 1500, 1800, 2100, 2400, 2700, 3000 nucleotides not encoding a stop codon.

In some embodiments, the polypeptide-encoding nucleotide sequence is at least 9, at least 12, at least 15, at least 18, at least 21, at least 24, at least 27, at least 30, at least 33, at least 36, at least 39, at least 42, at least 45, at least 48, at least 51, at least 54, at least 57, at least 60, at least 63, at least 66, at least 69, or at least 72 nucleotides in length and a multiple of 3 nucleotides in length. For example, the polypeptide-encoding nucleotide sequence may be 15, 18, 21, 24, 27, 30, 33, 36, 39, 42, 45, 48, 51, 54, 57, 60, 63, 66, 69, 72, 75, 90, 120, 150, 180, 210, 240, 270, 300, 450, 600, 750, 900, 1200, 1500, 1800, 2100, 2400, 2700, 3000 nucleotides in length. In some embodiments, the polypeptide-encoding nucleotide sequence is 18 nucleotides in length. The polypeptide coding nucleotide sequence may be at the 5′ upstream or at the 3′ downstream of the RNA sequence transcribed from a cDNA fragment sequence from a library of cDNA fragment sequences.

In certain embodiments, the polypeptide-encoding nucleotide sequence of the RNA transcript may encode a small soluble protein or soluble domain(s) of a protein which includes but is not limited to Titin 127, ubiquitin, Stefin A, 10FN-III, Ig-L filamin A, tenascin, Darpin, fibronectin, thioredoxin or any other small protein domain (derived from humans or any other species) which is highly soluble when expressed by in vitro translation or in E. Coli. In certain embodiments, the polypeptide-encoding nucleotide sequence may encode a polypeptide with an affinity tag. Such affinity tags include, but are not limited to, a hexa-histidine tag (SEQ ID NO: 15), a hemagglutinin (HA) tag, a Calmodulin tag, a FLAG tag, a Myc tag, a S tag, a Streptavidin tag, a SBP tag, a Softag 1, a Softag 3, a V5 tag, a Xpress tag, a Isopeptag, a SpyTag, a Biotin Carboxyl Carrier Protein (BCCP) tag, a GST tag, a fluorescent protein tag (e.g., a green fluorescent protein tag), a maltose binding protein tag, a Nus tag, a Strep-tag, a thioredoxin tag, a TC tag, a Ty tag, and the like. In certain embodiments, the polypeptide-encoding nucleotide encodes the polypeptide from the reading frame initiating at the first 5′ nucleotide of the nucleotide sequence.

In some embodiments, the adapter sequence is at least 9, at least 12, at least 15, at least 18, at least 21, at least 24, at least 27, at least 30, at least 33, at least 36, at least 39, at least 42, at least 45, at least 48, at least 51, at least 54, at least 57, at least 60, at least 63, at least 66, at least 69, or at least 72 nucleotides in length and a multiple of 3 nucleotides in length. For example, the adapter sequence may be 15, 18, 21, 24, 27, 30, 33, 36, 39, 42, 45, 48, 51, 54, 57, 60, 63, 66, 69, 72, 75, 90, 120, 150, 180, 210, 240, 270, 300, 450, 600, 750, 900, 1200, 1500, 1800, 2100, 2400, 2700, 3000 nucleotides in length. In certain embodiments, the adaptor sequence is at the 3′ downstream of the RNA sequence transcribed from a cDNA fragment sequence from a library of cDNA fragment sequences.

The splint polynucleotides described herein may comprise, in 3′ to 5′ order, a sequence complementary to the 3′ end of the polypeptide-encoding nucleotide sequence or to the adapter sequence, and a poly-T sequence. In certain embodiments, the splint polynucleotides may comprise a poly-T sequence of greater than 4, 5, 6, 7, 8, 9, 10, 11, 12, 13, 14, 15, 16, 17, 18, 19, 20 nucleotides.

The linker polynucleotides described herein may comprise, in 5′ to 3′ order a poly-dA sequence and a puromycin molecule. In certain embodiments, the linker polynucleotides may comprise a poly-dA sequence of greater than 4, 5, 6, 7, 8, 9, 10, 11, 12, 13, 14, 15, 16, 17, 18, 19, 20 nucleotides.

In certain embodiments, a ligation reaction is performed in the presence of T4 DNA ligase under conditions such that the 3′ end of the RNA transcripts is ligated to the 5′ end of the linker polynucleotides to generate puromycin-tagged RNA transcripts. Other methods that can ligate the 5′ end of the linker polynucleotide to the 3′ end of the RNA transcript can also be used.

In certain aspects, the methods of enriching a library of in-frame coding region fragments from a population of RNA transcripts described herein further comprise the step of generating the library of RNA transcripts prior to step (a) by performing a transcription reaction on a library of RNA expression constructs.

In some embodiments, each RNA expression construct in the library of RNA expression constructs comprises: (i) a transcription promoter; (ii) a translation initiation site; (iii) a cDNA fragment sequence from a library of cDNA fragment sequences; and (iv) a polypeptide coding nucleotide sequence lacking an in-frame stop codon in the reading frame initiating at the first 5′ nucleotide of the nucleotide sequence but containing stop codons in each of the other two reading frames. In certain embodiments, the translation initiation site comprises a Shine-Dalgarno sequence.

The transcription promoter of the RNA expression construct can be any promoter that is capable of initiating transcription of RNA from the DNA downstream of it. Such promoters include but are not limited to T7 promoter.

The translation start site of the RNA expression construct may comprise a start codon (e.g., ATG), a Shine-Dalgarno (SD) sequence and/or translational enhancers. The Shine-Dalgarno sequence is a ribosomal binding site that commonly presents in bacterial and archaeal messenger RNA and generally located around 8 bases upstream of the start codon AUG. The Shine-Dalgarno sequence may comprise AGGAGG, AGGAGGU, GAGG, ACAGGAGGCA (SEQ ID NO: 4), UAAGGAGGUG (SEQ ID NO: 5). Translational enhancer sequences are sequences upstream of the Shine-Dalgarno sequence which can further increase the amount of protein synthesis. The translational enhancer may comprise an A/U-rich enhancer, for example, 5′-GCUCUUUAACAAUUUAUCA-3′ (SEQ ID NO: 6), 5′-ACAUGGAUUC-3′ (SEQ ID NO: 7), 5′-UUAACUUUAA-3′ (SEQ ID NO: 8), 5′-UUAACGGGAA-3′ (SEQ ID NO: 9), 5′-AAAAAAAAAA-3′ (SEQ ID NO: 10), 5′-UUAACUUUAA-(A)₅-3′ (SEQ ID NO: 11), 5′-UUAACUUUAA-(A)₁₀-3′ (SEQ ID NO: 12), 5′-UUAACUUUAA-(A)₂₀-3′ (SEQ ID NO: 13), or 5′-UUAACUUUAA-(ACAUGGAUUC)₂-3′ (SEQ ID NO: 14). The translation start site may comprise a short (10-20 nucleotide) stretch of A residues between the translation enhancer sequence and the Shine-Dalgarno sequence to further improve translation efficiency.

In some embodiments, the translation initiation site of the RNA expression construct is followed by any multiple of 3 nucleotides not encoding a stop codon. For example, the translation initiation site may be followed by 0, 3, 6, 9, 12, 15, 18, 21, 24, 27, 30, 33, 36, 39, 42, 45, 48, 51, 54, 57, 60, 63, 66, 69, 72, 75, 90, 120, 150, 180, 210, 240, 270, 300, 450, 600, 750, 900, 1200, 1500, 1800, 2100, 2400, 2700, 3000 nucleotides not encoding a stop codon.

In some embodiments, the polypeptide-encoding nucleotide sequence is at least 9, at least 12, at least 15, at least 18, at least 21, at least 24, at least 27, at least 30, at least 33, at least 36, at least 39, at least 42, at least 45, at least 48, at least 51, at least 54, at least 57, at least 60, at least 63, at least 66, at least 69, or at least 72 nucleotides in length and a multiple of 3 nucleotides in length. For example, the polypeptide-encoding nucleotide sequence may be 15, 18, 21, 24, 27, 30, 33, 36, 39, 42, 45, 48, 51, 54, 57, 60, 63, 66, 69, 72, 75, 90, 120, 150, 180, 210, 240, 270, 300, 450, 600, 750, 900, 1200, 1500, 1800, 2100, 2400, 2700, 3000 nucleotides in length. The polypeptide coding nucleotide sequence may be at the 5′ upstream or at the 3′ downstream of the cDNA fragment sequence from a library of cDNA fragment sequences.

In certain embodiments, the polypeptide-encoding nucleotide sequence of the RNA expression construct may encode a small soluble protein or soluble domain(s) of a protein which includes but is not limited to Titin 127, ubiquitin, Stefin A, 10FN-III, Ig-L filamin A, Darpin, tenascin, fibronectin, thioredoxin or any other small protein domain (derived from humans or any other species) which is highly soluble when expressed by in vitro translation or in E. Coli. In certain embodiments, the polypeptide-encoding nucleotide sequence may encode a polypeptide with an affinity tag. Such affinity tags include, but are not limited to, a hexa-histidine tag (SEQ ID NO: 15), a hemagglutinin (HA) tag, a Calmodulin tag, a FLAG tag, a Myc tag, a S tag, a Strep tag, a SBP tag, a Softag 1, a Softag 3, a V5 tag, a Xpress tag, a Isopeptag, a SpyTag, a Biotin Carboxyl Carrier Protein (BCCP) tag, a GST tag, a fluorescent protein tag (e.g., a green fluorescent protein tag), a maltose binding protein tag, a Nus tag, a Strep-tag, a thioredoxin tag, a TC tag, a Ty tag, and the like. In certain embodiments, the polypeptide-encoding nucleotide encodes the polypeptide from the reading frame initiating at the first 5′ nucleotide of the nucleotide sequence.

In some embodiments, each RNA expression construct further comprises an adapter sequence which lacks stop codons in the reading frame beginning at the first 5′ nucleotide of the adapter sequence but contains stop codons in the other two reading frames.

In some embodiments, the adapter sequence is at least 9, at least 12, at least 15, at least 18, at least 21, at least 24, at least 27, at least 30, at least 33, at least 36, at least 39, at least 42, at least 45, at least 48, at least 51, at least 54, at least 57, at least 60, at least 63, at least 66, at least 69, or at least 72 nucleotides in length and a multiple of 3 nucleotides in length. For example, the adapter sequence may be 15, 18, 21, 24, 27, 30, 33, 36, 39, 42, 45, 48, 51, 54, 57, 60, 63, 66, 69, 72, 75, 90, 120, 150, 180, 210, 240, 270, 300, 450, 600, 750, 900, 1200, 1500, 1800, 2100, 2400, 2700, 3000 nucleotides in length. In certain embodiments, the adaptor sequence is at the 3′ downstream of the cDNA fragment sequence from a library of cDNA fragment sequences.

In certain embodiments, the RNA expression constructs are generated by PCR-based addition of the transcription promoter, the translation initiation site, the polypeptide-coding nucleotide sequence, and optionally the adapter sequence to a library of cDNA fragment sequences. The library of cDNA fragment sequences may be enriched for exome-containing cDNA fragments and/or mismatch-containing cDNA fragment sequences. In certain embodiments, the transcription of the RNA expression constructs is conducted in vitro in the presence of T7 polymerase. In certain embodiments, the translation initiation site comprises a Shine-Dalgarno sequence.

In certain aspects, provided herein are methods of enriching a library of in-frame coding region fragments from a population of cellular RNA fragments. Compared to methods described above for enriching a library of in-frame coding region fragments from a population of RNA transcripts, the methods of enriching a library of in-frame coding region fragments from a population of cellular RNA fragments further comprise steps of generating a population of RNA transcripts described herein from a population of cellular RNA fragments.

Such additional steps of generating a population of RNA transcripts from a population of cellular RNA fragments may comprise: (a) performing strand-specific random primed nucleic acid amplification reaction on a population of cellular RNA fragments to generate a population of cDNA fragments; (b) contacting the population of cDNA fragments with exome capture probes thereby enriching the population of cDNA fragments for exome-encoding cDNA fragments to generate a library of exome-enriched cDNA fragments; (c) generating RNA expression constructs from the library of exome-enriched cDNA fragments; and (d) performing a transcription reaction using the RNA expression constructs to generate a library of RNA transcripts.

The RNA expression constructs generated in the step (c) and the library of RNA transcripts generated in the step (d) may have the same structures as those described in the methods of enriching a library of in-frame coding region fragments from a population of RNA transcripts. In certain embodiments, the RNA expression constructs are generated by PCR-based addition of the transcription promoter, the translation initiation site, the polypeptide-coding nucleotide sequence, and optionally the adapter sequence to the library of exome-enriched cDNA fragments prepared from a population of cellular RNA fragments. In certain embodiments, the translation initiation site comprises a Shine-Dalgarno sequence.

In some embodiments, the methods of enriching a library of in-frame coding region fragments from a population of RNA transcripts or from a population of cellular RNA fragments described herein further comprise affinity purifying the protein-linked RNA complexes using a reagent that binds to the polypeptide encoded by the polypeptide-encoding nucleotide sequence. The reagent that binds to the polypeptide may be an antibody that specifically binds to the affinity tag linked to the polypeptide, or an antibody that specifically binds to the polypeptide itself. Antibodies that specifically bind to affinity tags are well known in the art and commercially available. In some aspects, provided herein is a library of purified polypeptide-linked RNA complexes generated according to the methods described herein.

In some embodiments, the methods of enriching a library of in-frame coding region fragments from a population of RNA transcripts or from a population of cellular RNA fragments described herein further comprise performing an RT-PCR amplification reaction on the purified protein-linked RNA complexes to generate an amplification product comprising an amplified DNA copy of the cDNA fragment sequence. In certain embodiments, the PCR reaction is conducted with strand-specific cloning primers such that the amplification products can be readily cloned into a vector. In some aspects, provided herein are the amplification products generated with the methods described herein.

In certain aspects, provided herein are methods of enriching a library of in-frame coding region fragments from a population of cellular RNA fragments, the method comprising: the method comprising: (a) performing strand-specific random primed nucleic acid amplification reaction on a population of cellular RNA fragments to generate a population of cDNA fragments; (b) inserting the population of cDNA fragments into cloning vectors to generate a library of DNA constructs, wherein each DNA construct comprises, in 5′ to 3′ order: (i) a promoter; (ii) a translation initiation site; (iii) a polypeptide-encoding nucleotide sequence which is a multiple of 3 nucleotides in length and lacks an in-frame stop codon in the reading frame initiating at the first 5′ nucleotide of the nucleotide sequence; (iv) one cDNA fragment from the population of cDNA fragments; and (v) a membrane-presenting protein-encoding sequence, (c) transforming the library of DNA constructs into cells, (d) incubating the cells under conditions such that they express the DNA constructs; (e) purifying (e.g., affinity purifying) the cells that express a complete fusion protein comprising the polypeptide encoded by the polypeptide-encoding nucleotide sequence, the polypeptide encoded by the cDNA fragment, and the membrane-presenting protein using a reagent that binds to the polypeptide encoded by the polypeptide-encoding nucleotide sequence; and (f) recovering in-frame cDNA fragment sequences from the purified cells (e.g., by PCR amplification), thereby enriching a library of in-frame coding region fragments from a population of RNA transcripts.

In some embodiments, the step (a) further comprises contacting the population of cDNA fragments with exome capture probes thereby enriching the population of cDNA fragments for exome-encoding cDNA fragments to generate a library of exome-enriched cDNA fragments. The library of exome-enriched cDNA fragments may then be used for the following steps.

In some embodiments, the promoter of the DNA construct is a promoter that is capable of driving expression of genes in bacteria (e.g., E. coli). Such promoters include but are not limited to bacteriophage T7 promotor.

The translation start site of the DNA construct may comprise a start codon (e.g., ATG), a Shine-Dalgarno (SD) sequence and/or translational enhancers. The Shine-Dalgarno sequence is a ribosomal binding site that commonly presents in bacterial and archaeal messenger RNA and generally located around 8 bases upstream of the start codon AUG. The Shine-Dalgarno sequence may comprise AGGAGG, AGGAGGU, GAGG, ACAGGAGGCA (SEQ ID NO: 4), UAAGGAGGUG (SEQ ID NO: 5). Translational enhancer sequences are sequences upstream of the Shine-Dalgarno sequence which can further increase the amount of protein synthesis. The translational enhancer may comprise an A/U-rich enhancer, for example, 5′-GCUCUUUAACAAUUUAUCA-3′ (SEQ ID NO: 6), 5′-ACAUGGAUUC-3′ (SEQ ID NO: 7), 5′-UUAACUUUAA-3′ (SEQ ID NO: 8), 5′-UUAACGGGAA-3′ (SEQ ID NO: 9), 5′-AAAAAAAAAA-3′ (SEQ ID NO: 10), 5′-UUAACUUUAA-(A)₅-3′ (SEQ ID NO: 11), 5′-UUAACUUUAA-(A)₁₀-3′ (SEQ ID NO: 12), 5′-UUAACUUUAA-(A)₂₀-3′ (SEQ ID NO: 13), or 5′-UUAACUUUAA-(ACAUGGAUUC)₂-3′ (SEQ ID NO: 14). The translation start site may comprise a short (10-20 nucleotide) stretch of A residues between the translation enhancer sequence and the Shine-Dalgarno sequence to further improve translation efficiency.

In some embodiments, the translation initiation site of the DNA construct is followed by any multiple of 3 nucleotides not encoding a stop codon. For example, the translation initiation site may be followed by 0, 3, 6, 9, 12, 15, 18, 21, 24, 27, 30, 33, 36, 39, 42, 45, 48, 51, 54, 57, 60, 63, 66, 69, 72, 75, 90, 120, 150, 180, 210, 240, 270, 300, 450, 600, 750, 900, 1200, 1500, 1800, 2100, 2400, 2700, 3000 nucleotides not encoding a stop codon.

In some embodiments, the polypeptide-encoding nucleotide sequence is at least 9, at least 12, at least 15, at least 18, at least 21, at least 24, at least 27, at least 30, at least 33, at least 36, at least 39, at least 42, at least 45, at least 48, at least 51, at least 54, at least 57, at least 60, at least 63, at least 66, at least 69, or at least 72 nucleotides in length and a multiple of 3 nucleotides in length. For example, the polypeptide-encoding nucleotide sequence may be 15, 18, 21, 24, 27, 30, 33, 36, 39, 42, 45, 48, 51, 54, 57, 60, 63, 66, 69, 72, 75, 90, 120, 150, 180, 210, 240, 270, 300, 450, 600, 750, 900, 1200, 1500, 1800, 2100, 2400, 2700, 3000 nucleotides in length.

In certain embodiments, the polypeptide-encoding nucleotide sequence of the DNA construct may encode a small soluble protein or soluble domain(s) of a protein which includes but is not limited to Titin 127, ubiquitin, Stefin A, 10FN-III, Ig-L filamin A, Darpin, tenascin, fibronectin, thioredoxin or any other small protein domain (derived from humans or any other species) which is highly soluble when expressed by in vitro translation or in E. Coli. In certain embodiments, the polypeptide-encoding nucleotide sequence may encode a polypeptide with an affinity tag. Such affinity tags include, but are not limited to, a hexa-histidine tag (SEQ ID NO: 15), a hemagglutinin (HA) tag, a Calmodulin tag, a FLAG tag, a Myc tag, a S tag, a Strep tag, a SBP tag, a Softag 1, a Softag 3, a V5 tag, a Xpress tag, a Isopeptag, a SpyTag, a Biotin Carboxyl Carrier Protein (BCCP) tag, a GST tag, a fluorescent protein tag (e.g., a green fluorescent protein tag), a maltose binding protein tag, a Nus tag, a Strep-tag, a thioredoxin tag, a TC tag, a Ty tag, and the like. In certain embodiments, the polypeptide-encoding nucleotide encodes the polypeptide from the reading frame initiating at the first 5′ nucleotide of the nucleotide sequence.

In certain embodiments, the membrane-presenting protein-encoding sequence may encode any membrane-presenting protein that allows the insertion of the translated protein into the outer cellular membrane and the exposure of the polypeptide encoded by the polypeptide-encoding nucleotide sequence on the outer surface of the cell. In specific embodiments, the membrane-presenting protein-encoding sequence encodes a bacterial membrane-presenting protein, such as the adhesion-involved-in-diffuse-adherence (AIDA-I) auto-transporter, which allows the insertion of the translated protein into the outer bacterial membrane and exposure of the peptide sequence encoded by the polypeptide-encoding nucleotide sequence on the outer surface of the bacterial cell.

In some embodiment, the cells are eukaryotic cells (e.g., mammalian cells). In some embodiments, the cells are prokaryotic cells (e.g., bacteria). In certain embodiments, the bacterial cells (e.g., E. Coli) are from a strain that is able to specifically control the expression of the T7 RNA polymerase. Such bacterial strain includes but is not limited to the strain that bears the gene of the T7 RNA polymerase under the control of the araBAD promotor such that a small molecule (e.g., arabinose) can be added to the bacteria (e.g., E. coli) culture to induces expression of the T7 RNA polymerase. The T7 RNA Polymerase may then induce the expression of the DNA construct comprising the population of cDNA fragments and insertion of the translated protein into the outer membrane of the bacteria (e.g., E. coli).

In certain embodiments, the cells are transfected or transformed with the DNA constructs at a ratio such that each cell has no more than one (e.g., 0 or 1) DNA construct.

In certain embodiments, the reagent used for affinity purification binds to the polypeptide encoded by the polypeptide-encoding sequence. Reagent that binds to the polypeptide may be an antibody that specifically binds to the affinity tag linked to the polypeptide, or an antibody that specifically binds to the polypeptide itself.

In certain embodiments, the membrane-presenting protein-encoding sequence encodes a membrane-presenting protein that is not expressed endogenously by the cells. In such cases, the DNA constructs need not comprise the polypeptide-encoding nucleotide sequence, and the affinity purification can use a reagent that binds to the membrane-presenting protein.

In some embodiments, methods other than affinity purification may be used for enriching the cells expressing a complete fusion protein comprising the polypeptide encoded by the in-frame cDNA fragment. For example, in some embodiments the polypeptide-encoding nucleotide sequence encodes a c-terminal selection marker. In some embodiments, the c-terminal selection marker is a drug resistance gene (e.g., an antibiotic resistance gene), and cells expressing a complete fusion protein comprising the polypeptide encoded by the in-frame cDNA fragment and the drug resistance gene can be enriched by adding the drug to the cell culture. In certain embodiments, the c-terminal selection marker is an protein that allows for cell survival in the absence of a cell culture medium component and cells expressing a complete fusion protein comprising the polypeptide encoded by the in-frame cDNA fragment and the c-terminal selection marker can be enriched by withdrawing the component from the cell culture medium. In some embodiments, the c-terminal selection marker is a fluorescent protein, and cells expressing a complete fusion protein comprising the polypeptide encoded by the in-frame cDNA fragment and the drug resistant gene can be enriched by FACS.

In certain embodiments, the PCR amplification reaction in the step (f) is conducted with strand-specific cloning primers such that the amplification products can be readily cloned into a vector. In some aspects, provided herein are the amplification products generated with the methods described herein.

The population of cellular RNA fragments may be prepared from a sample, such as a tumor sample, a normal tissue sample, a diseased tissue sample; a fresh sample, a frozen sample, and/or a paraffin embedded (FFPE) sample. In certain embodiments, the sample is a paraffin embedded (FFPE) tissue or tumor sample. The sample may be obtained from a subject (e.g., a human, preferably a cancer patient) and will be prepared specifically for each subject. The sample may aso be prepared for a subject and used for different subjects. The total RNA or mRNA from these samples may be isolated and fragmented to appropriate sizes. The cellular RNA fragments in the population of cellular RNA fragments may be of between 150 and 250 nt in length. For example, the cellular RNA fragments in the population of cellular RNA fragments may be of about 150 nt, about 160 nt, about 170 nt, about 180 nt, about 190 nt, about 200 nt, about 210 nt, about 220 nt, about 230 nt, about 240 nt, about 250 nt in length. In certain embodiments, the cellular RNA fragments in the population of cellular RNA fragments are of about 200 nt in length.

The strand-specific random primed nucleic acid amplification reaction to generate the population of cDNA fragments may be performed using any standard protocol such as the Illiumina TruSeq Stranded Total RNA protocol.

In some embodiments, the methods of enriching a library of in-frame coding region fragments described herein further comprise contacting the population of cDNA fragments with a MutS protein and recovering those cDNA fragments that bind to the MutS protein, thereby enriching the population of cDNA fragments for mismatch-containing cDNA fragments due to either mutations or to single nucleotide polymorphisms.

In some embodiments, the methods of enriching a library of in-frame coding region fragments described herein further comprise contacting the library of exome-enriched cDNA fragments with a MutS protein and recovering those cDNA fragments that bind to the MutS protein, thereby enriching the library of exome-enriched cDNA fragments for mismatch-containing cDNA fragments due to either mutations or to single nucleotide polymorphisms.

In some embodiments, the methods of enriching a library of in-frame coding region fragments described herein further comprise contacting the in-frame enriched amplification products with a MutS protein and recovering those in-frame cDNA fragments that bind to the MutS protein, thereby enriching the in-frame enriched amplification products for mismatch-containing cDNA fragments due to either mutations or to single nucleotide polymorphisms.

The exome capture probes used to generate a library of exome-enriched cDNA fragments may be standard exome capture probes that are designed based on reference genome sequences and therefore capture primarily coding region exons from all known CDS. Alternatively, the exome capture probes may be designed based on the known locations and frequencies of SNPs such that these exome capture probes are designed around the locations of these SNPs to reduce the MutS enrichment of SNPs. Additional considerations for designing exome capture probes are described herein in example 1.

The term “exome” refers to a complete exome or any desired portion of the complete exome based on the cell types, the tissues and the disease being studied, and the level of RNA transcription desired, etc.

### Methods of Making a Tumor Vaccine

In certain aspect, provided herein are methods of making a tumor vaccine using one or more of the amplification products generated with the methods described herein. One of skill in the art from this disclosure and the knowledge in the art will appreciate that there are a variety of ways in which to produce such tumor vaccine. In general, such tumor vaccine may be produced either in vitro or in vivo. The one or more of the amplification products comprising the in-frame cDNA fragment sequences may be expressed in vitro to produce one or more tumor specific peptides or polypeptides, which may then be formulated into a personalized tumor vaccine or immunogenic composition and administered to a subject. As described in further detail herein, such in vitro production may occur by a variety of methods known to one of skill in the art such as, for example, expression of one or more of the amplification products in any of a variety of bacterial, eukaryotic, or viral recombinant expression systems, followed by purification of the expressed peptide/polypeptide. Alternatively, tumor vaccine may be produced in vivo by inserting one or more of the amplification products into an expression vector and then introducing such expression vectors into a subject, whereupon the encoded tumor vaccine is expressed. The methods of in vitro and in vivo production of tumor vaccine is also further described herein as it relates to pharmaceutical compositions and methods of delivery.

In certain embodiments, to make a tumor vaccine, the amplification product generated with the methods described herein is inserted into a vector to generate vectors comprising sequences of the in-frame cDNA fragments. These vectors may be cloning vectors, expression vectors, or vaccine-coding vectors.

Expression vectors for different cell types are well known in the art and can be selected without undue experimentation. Generally, the amplification product is inserted into an expression vector, such as a plasmid, in proper orientation and correct reading frame for expression. If necessary, the amplification product may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the amplified products, as well as host cells containing the expression vectors, are also contemplated. One or more amplified products of the invention may be encoded by a single expression vector.

In some embodiments, the amplification product is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express cDNA fragment sequences encoding the tumor specific neoantigenic peptides. Recombinant expression vectors are replicable DNA constructs which have synthetic or cDNA-derived DNA fragments encoding a tumor specific neoantigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription.

The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine (SEQ ID NO: 15), maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents

In some embodiments, the vectors may be subjected to an in vitro translation reaction to generate the tumor vaccine. Many exemplary systems exist that one skilled in the art could utilize (e.g., Retic Lysate IVT Kit, Life Technologies, Waltham, Mass.).

The present invention also contemplates the use of nucleic acid molecules as vehicles for delivering neoantigenic peptides/polypeptides to the subject in need thereof, in vivo or ex vivo, in the form of, e.g., DNA/RNA vaccines (see, e.g., WO2012/159643, and WO2012/159754, hereby incorporated by reference in their entirety).

In one embodiment, vectors (e.g., expression vectors) comprising the in-frame cDNA fragment sequences may be administered to a patient in need thereof to produce a tumor vaccine in vivo. These are vectors which usually consist of a strong viral promoter to drive the in vivo transcription and translation of the gene (or complementary DNA) of interest (Mor, et al., (1995). The Journal of Immunology 155 (4): 2039-2046). Intron A may sometimes be included to improve mRNA stability and hence increase protein expression (Leitner et al. (1997).The Journal of Immunology 159 (12): 6112-6119). Plasmids also include a strong polyadenylation/transcriptional termination signal, such as bovine growth hormone or rabbit beta-globulin polyadenylation sequences (Alarcon et al., (1999). Adv. Parasitol. Advances in Parasitology 42: 343-410; Robinson et al., (2000). Adv. Virus Res. Advances in Virus Research 55: 1-74; Bohmet al., (1996). Journal of Immunological Methods 193 (1): 29-40.). Multicistronic vectors are sometimes constructed to express more than one immunogen, or to express an immunogen and an immunostimulatory protein (Lewis et al., (1999). Advances in Virus Research (Academic Press) 54: 129-88).

Because the vector is the “vehicle” from which the tumor vaccine is expressed, optimizing vector design for maximal protein expression is essential (Lewis et al., (1999). Advances in Virus Research (Academic Press) 54: 129-88). Another consideration is the choice of promoter. Such promoters may be the SV40 promoter or Rous Sarcoma Virus (RSV).

Vectors may be introduced into animal tissues by a number of different methods. The two most popular approaches are injection of DNA in saline, using a standard hypodermic needle, and gene gun delivery. A schematic outline of the construction of a DNA vaccine vector and its subsequent delivery by these two methods into a host is illustrated at Scientific American (Weiner et al., (1999) Scientific American 281 (1): 34-41). Injection in saline is normally conducted intramuscularly (IM) in skeletal muscle, or intradermally (ID), with DNA being delivered to the extracellular spaces. This can be assisted by electroporation by temporarily damaging muscle fibres with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected(Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors.

The method of delivery determines the dose of DNA required to raise an effective immune response. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshiaet al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In certain embodiments, the vaccine-encoding vectors disclosed herein can be used in ex vivo immune therapies. For example, in some embodiments, the vaccine-encoding vectors can be transfected or transduced into antigen presenting cells (e.g., dendritic cells) In certain embodiments these antigen presenting cells are then administered to a subject. In some embodiments, these antigen presenting cells are used to activate T cells (e.g., autologous T cells, syngeneic T cells) in vitro, which are then administered to the subject.

In one embodiment, a tumor vaccine or immunogenic composition may include separate DNA plasmids encoding, for example, one or more neoantigenic peptides/polypeptides as identified according to the invention. As discussed herein, the exact choice of expression vectors can depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection.

Alternatively, the in-frame enrich RNA library can be transfected or electroporated into cells in vitro, or delivered to a subject in vivo directly. Self-replicating RNAs may be used to generate the RNA vaccines. The RNA vaccine can be delivered to a subject using a number of methods, e.g., subcutaneous, intramuscular, or intravenous injection, topical application to the skin, or via a nasal spray. The RNA vaccine may also be delivered using lipid nanoparticles or RNA viruses. Typical RNA viruses used as vectors include but are not limited to retroviruses, lentiviruses, alphaviruses and rhabdoviruses.

Tumor vaccine of the invention may be encoded and expressed in vivo using a viral based system (e.g., an adenovirus system, an adeno associated virus (AAV) vector, a poxvirus, or a lentivirus). In one embodiment, the tumor vaccine or immunogenic composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan 15;207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. Patent application No. 20080254008, hereby incorporated by reference).

Among vectors that may be used in the practice of the invention, integration in the host genome of a cell is possible with retrovirus gene transfer methods, often resulting in long term expression of the inserted transgene. In a preferred embodiment the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), Simian Immuno deficiency virus (SW), human immuno deficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700). Zou et al. administered about 10 μl of a recombinant lentivirus having a titer of 1×10⁹ transducing units (TU)/ml by an intrathecal catheter. These sort of dosages can be adapted or extrapolated to use of a retroviral or lentiviral vector in the present invention.

Also useful in the practice of the invention is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience (interscience.wiley.com). DOI: 10.1002/jgm.845). The vectors may have cytomegalovirus (CMV) promoter driving expression of the target gene. Accordingly, the invention contemplates amongst vector(s) useful in the practice of the invention: viral vectors, including retroviral vectors and lentiviral vectors.

Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: Synapsinl for all neurons, CaMKllalpha for excitatory neurons, GAD67 or GAD65 or VGAT for GABAergic neurons, etc. Promoters used to drive RNA synthesis can include: Pol III promoters such as U6 or H1. The use of a Pol II promoter and intronic cassettes can be used to express guide RNA (gRNA).

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10¹⁴ functional AAV/ml solution. The dosage may be adjusted to balance the therapeutic benefit against any side effects. In an embodiment herein, the AAV dose is generally in the range of concentrations of from about 1×10⁵ to 1×10¹⁴ genomes AAV, from about 1×10⁸ to 1×10¹⁴ genomes AAV, from about 1×10¹⁰ to about 5×10¹³ genomes, or about 1×10¹¹ to about 1×10¹³ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. In a preferred embodiment, AAV is used with a titer of about 2×10¹³ viral genomes/milliliter, and each of the striatal hemispheres of a mouse receives one 500 nanoliter injection. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In another embodiment effectively activating a cellular immune response for a tumor vaccine or immunogenic composition can be achieved by expressing the relevant antigens in a vaccine or immunogenic composition in a non-pathogenic microorganism. Well-known examples of such microorganisms are Mycobacterium bovis BCG, Salmonella and Pseudomona (See, U.S. Pat. No. 6,991,797, hereby incorporated by reference in its entirety).

In another embodiment a Poxvirus is used in the tumor vaccine or immunogenic composition. These include orthopoxvirus, avipox, vaccinia, MVA, NYVAC, canarypox, ALVAC, fowlpox, TROVAC, etc. (see e.g., Verardiet al., Hum Vaccin Immunother. 2012 July; 8(7):961-70; and Moss, Vaccine. 2013; 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

In another embodiment the vaccinia virus is used in the tumor vaccine or immunogenic composition to express a neoantigen. (Rolph et al., Recombinant viruses as vaccines and immunological tools. Curr Opin Immunol 9:517-524, 1997). The recombinant vaccinia virus is able to replicate within the cytoplasm of the infected host cell and the polypeptide of interest can therefore induce an immune response. Moreover, Poxviruses have been widely used as vaccine or immunogenic composition vectors because of their ability to target encoded antigens for processing by the major histocompatibility complex class I pathway by directly infecting immune cells, in particular antigen-presenting cells, but also due to their ability to self-adjuvant.

In another embodiment ALVAC is used as a vector in a tumor vaccine or immunogenic composition. ALVAC is a canarypox virus that can be modified to express foreign transgenes and has been used as a method for vaccination against both prokaryotic and eukaryotic antigens (Horig H, Lee DS, Conkright W, et al. Phase I clinical trial of a recombinant canarypoxvirus (ALVAC) vaccine expressing human carcinoembryonic antigen and the B7.1 co-stimulatory molecule. Cancer Immunol Immunother 2000;49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000;6:2219-28; Musey L, Ding Y, Elizaga M, et al. HIV-1 vaccination administered intramuscularly can induce both systemic and mucosal T cell immunity in HIV-1-uninfected individuals. J Immunol 2003;171:1094-101; Paoletti E. Applications of pox virus vectors to vaccination: an update. Proc Natl Acad Sci U S A 1996;93:11349-53; U.S. Pat. No. 7,255,862). In a phase I clinical trial, an ALVAC virus expressing the tumor antigen CEA showed an excellent safety profile and resulted in increased CEA-specific T-cell responses in selected patients; objective clinical responses, however, were not observed (Marshall J L, Hawkins M J, Tsang K Y, et al. Phase I study in cancer patients of a replication-defective avipox recombinant vaccine that expresses human carcinoembryonic antigen. J Clin Oncol 1999; 17:332-7).

In another embodiment a Modified Vaccinia Ankara (MVA) virus may be used as a viral vector for a tumor vaccine or immunogenic composition. MVA is a member of the Orthopoxvirus family and has been generated by about 570 serial passages on chicken embryo fibroblasts of the Ankara strain of Vaccinia virus (CVA) (for review see Mayr, A., et al., Infection 3, 6-14, 1975). As a consequence of these passages, the resulting MVA virus contains 31 kilobases less genomic information compared to CVA, and is highly host-cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol Immunother. January 2012; 61(1): 19-29). Methods to make and use recombinant MVA has been described (e.g., see U.S. Pat. Nos. 8,309,098 and 5,185,146 hereby incorporated in its entirety).

In another embodiment the modified Copenhagen strain of vaccinia virus, NYVAC and NYVAC variations are used as a vector (see U.S. Pat. No. 7,255,862; PCT WO 95/30018; U.S. Pat. Nos. 5,364,773 and 5,494,807, hereby incorporated by reference in its entirety).

In one embodiment recombinant viral particles of the vaccine or immunogenic composition are administered to patients in need thereof. The vaccine or immunogenic composition can be administered in any suitable amount to achieve expression at these dosage levels. The viral particles can be administered to a patient in need thereof or transfected into cells in an amount of about at least 10^{3.5} pfu; thus, the viral particles are preferably administered to a patient in need thereof or infected or transfected into cells in at least about 10⁴ pfu to about 10⁶ pfu; however, a patient in need thereof can be administered at least about 10⁸ pfu such that a more preferred amount for administration can be at least about 10⁷ pfu to about 10⁹ pfu. Doses as to NYVAC are applicable as to ALVAC, MVA, MVA-BN, and avipoxes, such as canarypox and fowlpox.

### Pharmaceutical Compositions/Methods of Delivery

In certain aspects, provided herein are pharmaceutical compositions comprising an amplification product comprising an in-frame cDNA fragment sequence produced with the methods described herein. In certain aspects, provided herein are pharmaceutical compositions comprising a vector comprising an in-frame cDNA fragment sequence produced with the methods described herein. In some embodiments, the pharmaceutical compositions provided herein further comprise a pharmaceutically acceptable carrier.

In certain embodiments, the pharmaceutical compositions are for use in generating tumor vaccine. In certain embodiments, the pharmaceutical compositions are for use in treating cancer.

The present invention is also directed to pharmaceutical compositions comprising an effective amount of a tumor vaccine produced with the methods described herein, optionally in combination with a pharmaceutically acceptable carrier, excipient or additive.

“Pharmaceutically acceptable carrier” refers to a substance that aids the administration of an active agent to and absorption by a subject and can be included in the compositions described herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylase or starch, fatty acid esters, hydroxymethy cellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions described herein. One of skill in the art will recognize that other pharmaceutical excipients are useful.

While the tumor vaccine can be administered as the sole active pharmaceutical agent, they can also be used in combination with one or more other agents and/or adjuvants. When administered as a combination, the therapeutic agents can be formulated as separate compositions that are given at the same time or different times, or the therapeutic agents can be given as a single composition.

The compositions may be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compositions of the invention can be used to treat diseases and disease conditions that are acute, and may also be used for treatment of chronic conditions. In particular, the compositions of the invention are used in methods to treat or prevent a tumor. In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient's life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

The tumor vaccine may be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

Surgical resection uses surgery to remove abnormal tissue in cancer, such as mediastinal, neurogenic, or germ cell tumors, or thymoma. In certain embodiments, administration of the tumor vaccine or immunogenic composition is initiated 1, 2, 3, 4, 5, 6, 7, 8, 9, 10, 11, 12, 13, 14, 15 or more weeks after tumor resection. Preferably, administration of the tumor vaccine or immunogenic composition is initiated 1,2,3,4, 5, 6, 7, 8, 9, 10, 11 or 12 weeks after tumor resection. In some embodiments, the tumor may not be totally resected and the administration of the tumor vaccine occurs while the tumor is still present in the patient.

Prime/boost regimens refer to the successive administrations of a vaccine or immunogenic or immunological compositions. In certain embodiments, administration of the tumor vaccine or immunogenic composition is in a prime/boost dosing regimen, for example administration of the tumor vaccine or immunogenic composition at weeks 1, 2, 3 or 4 as a prime and administration of the tumor vaccine or immunogenic composition is at months 2, 3 or 4 as a boost. In another embodiment heterologous prime-boost strategies are used to elicit a greater cytotoxic T-cell response (see Schneider et al., Induction of CD8+T cells using heterologous prime-boost immunization strategies, Immunological Reviews Volume 170, Issue 1, pages 29-38, August 1999). In another embodiment DNA encoding tumor vaccine is used to prime followed by a protein boost. In another embodiment protein is used to prime followed by boosting with a virus encoding the tumor vaccine. In another embodiment a virus encoding the tumor vaccine is used to prime and another virus is used to boost. In another embodiment protein is used to prime and DNA is used to boost. In a preferred embodiment a DNA vaccine or immunogenic composition is used to prime a T-cell response and a recombinant viral vaccine or immunogenic composition is used to boost the response. In another preferred embodiment a viral vaccine or immunogenic composition is co-administered with a protein or DNA vaccine or immunogenic composition to act as an adjuvant for the protein or DNA vaccine or immunogenic composition. The patient can then be boosted with either the viral vaccine or immunogenic composition, protein, or DNA vaccine or immunogenic composition (see Hutchings et al., Combination of protein and viral vaccines induces potent cellular and humoral immune responses and enhanced protection from murine malaria challenge. Infect Immun. 2007 December; 75(12):5819-26. Epub 2007 Oct. 1).

The pharmaceutical compositions can be processed in accordance with conventional methods of pharmacy to produce medicinal agents for administration to patients in need thereof, including humans and other mammals.

The tumor vaccine generated with the methods described herein may contain one or more neoantigens. In certain embodiments, the pharmaceutical composition further comprises an immunomodulator or adjuvant. In certain embodiments, the immunodulator or adjuvant is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In certain embodiments, the immunomodulator or adjuvant comprises poly-ICLC.

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine or immunogenic composition of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5,6-Dimethylxanthenone-4-Acetic Acid, Journal of Immunology, 190:5216-25 and Kim et al. (2013) Anticancer Flavonoids are Mouse-Selective STING Agonists, 8:1396-1401).

The tumor vaccine or immunological composition may also include an adjuvant compound chosen from the acrylic or methacrylic polymers and the copolymers of maleic anhydride and an alkenyl derivative. It is in particular a polymer of acrylic or methacrylic acid cross-linked with a polyalkenyl ether of a sugar or polyalcohol (carbomer), in particular cross-linked with an allyl sucrose or with allylpentaerythritol. It may also be a copolymer of maleic anhydride and ethylene cross-linked, for example, with divinyl ether (see U.S. Pat. No. 6,713,068 hereby incorporated by reference in its entirety).

Pharmaceutical compositions comprise the herein-described tumor vaccine in a therapeutically effective amount for treating diseases and conditions (e.g., a tumor), which have been described herein, optionally in combination with a pharmaceutically acceptable additive, carrier and/or excipient. One of ordinary skill in the art from this disclosure and the knowledge in the art will recognize that a therapeutically effective amount of one of more compounds according to the present invention may vary with the condition to be treated, its severity, the treatment regimen to be employed, the pharmacokinetics of the agent used, as well as the patient (animal or human) treated.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., ocular, oral, topical or parenteral, including gels, creams ointments, lotions and time released implantable preparations, among numerous others. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques.

The active compound is included in the pharmaceutically acceptable carrier or diluent in an amount sufficient to deliver to a patient a therapeutically effective amount for the desired indication, without causing serious toxic effects in the patient treated.

Oral compositions generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material herein discussed, a liquid carrier such as a fatty oil. In addition, dosage unit forms can contain various other materials which modify the physical form of the dosage unit, for example, coatings of sugar, shellac, or enteric agents.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Methods of formulating such slow or controlled release compositions of pharmaceutically active ingredients, are known in the art and described in several issued US Patents, some of which include, but are not limited to, U.S. Pat. Nos. 3,870,790; 4,226,859; 4,369,172; 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof may also be administered as a component of an elixir, suspension, syrup, wafer, chewing gum or the like. A syrup may contain, in addition to the active compounds, sucrose or fructose as a sweetening agent and certain preservatives, dyes and colorings and flavors.

Solutions or suspensions used for ocular, parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose.

In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS), and 5% dextrose in water (D5W) or 10% trehalose or 10% sucrose. In certain embodiments, the aqueous solvent further comprises dimethyl sulfoxide (DMSO), e.g., in an amount of about 1-4%, or 1-3%. In certain embodiments, the pharmaceutically acceptable carrier is isotonic (i.e., has substantially the same osmotic pressure as a body fluid such as plasma).

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations are within the ambit of the skilled artisan in view of this disclosure and the knowledge in the art.

A skilled artisan from this disclosure and the knowledge in the art recognizes that in addition to tablets, other dosage forms can be formulated to provide slow or controlled release of the active ingredient. Such dosage forms include, but are not limited to, capsules, granulations and gel-caps.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier usually comprises sterile water or aqueous sodium chloride solution, though other ingredients including those which aid dispersion may be included. Of course, where sterile water is to be used and maintained as sterile, the compositions and carriers are also sterilized. Injectable suspensions may also be prepared, in which case appropriate liquid carriers, suspending agents and the like may be employed.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include oral, topical, eye or ocular, parenteral, intramuscular, intravenous, sub-cutaneous, transdermal (which may include a penetration enhancement agent), buccal and suppository administration, among other routes of administration, including through an eye or ocular route.

The tumor vaccine or immunogenic composition may be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

The tumor vaccine may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

### Therapeutic Methods

The present invention provides methods of inducing a tumor specific immune response in a subject, vaccinating against a tumor, treating and or alleviating a symptom of cancer in a subject by administering the subject a tumor vaccine or a vector encoding a tumor vaccine generated according to the methods described herein.

According to the invention, the herein-described tumor vaccine or vector encoding a tumor vaccine may be used for a patient that has been diagnosed as having cancer, or at risk of developing cancer.

Cancers that can be treated using this tumor vaccine or vector encoding a tumor vaccine may include among others cases which are refractory to treatment with other chemotherapeutics. The term “refractory, as used herein refers to a cancer (and/or metastases thereof), which shows no or only weak antiproliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The tumor vaccine or vector encoding a tumor vaccine described herein is also applicable to the treatment of patients in need thereof who have not been previously treated.

The tumor vaccine or vector encoding a tumor vaccine described herein is also applicable where the subject has no detectable tumor but is at high risk for disease recurrence.

Also of special interest is the treatment of patients in need thereof who have undergone Autologous Hematopoietic Stem Cell Transplant (AHSCT), and in particular patients who demonstrate residual disease after undergoing AHSCT. The post-AHSCT setting is characterized by a low volume of residual disease, the infusion of immune cells to a situation of homeostatic expansion, and the absence of any standard relapse-delaying therapy. These features provide a unique opportunity to use the described neoplastic vaccine or immunogenic composition to delay disease relapse.

In certain embodiments, the pharmaceutical compositions, tumor vaccine or vector encoding a tumor vaccine described herein can be administered in conjunction with any other conventional anti-cancer treatment, such as, for example, radiation therapy and surgical resection of the tumor. These treatments may be applied as necessary and/or as indicated and may occur before, concurrent with or after administration of the pharmaceutical compositions, tumor vaccines, vectors coding tumor vaccines, dosage forms, and kits described herein.

The effective dose of tumor vaccine or vector encoding a tumor vaccine described herein is the amount of the tumor vaccine or vector encoding a tumor vaccine that is effective to achieve the desired therapeutic response for a particular patient, composition, and mode of administration, with the least toxicity to the patient. The effective dosage level can be identified using the methods described herein and depends upon a variety of pharmacokinetic factors including the activity of the particular compositions administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. In general, an effective dose of a cancer therapy is the amount of the therapeutic agent which is the lowest dose effective to produce a therapeutic effect. Such an effective dose generally depends upon the factors described above.

Examples of routes of administration include oral administration, rectal administration, topical administration, inhalation (nasal) or injection. Administration by injection includes intravenous (IV), intralesional, peritumoral, intramuscular (IM), and subcutaneous (SC) administration. The compositions described herein can be administered in any form by any effective route, including but not limited to oral, parenteral, enteral, intravenous, intratumoral, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In some embodiments, the pharmaceutical compositions, tumor vaccines, or vaccine-coding vectors described herein are administered orally, rectally, topically, intravesically, by injection into or adjacent to a draining lymph node, intravenously, by inhalation or aerosol, or subcutaneously. In some embodiments, the administration is parenteral administration (e.g., subcutaneous administration). The administration may be an intratumoral injection or a peritumoral injection.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, tumor dimensions and general health, the particular microorganism to be administered, duration and route of administration, the kind and stage of the disease, for example, tumor size, and other compounds such as drugs being administered concurrently.

The methods of treatment described herein may be suitable for the treatment of a primary tumor, a secondary tumor or metastasis, as well as for recurring tumors or cancers. The dose of the pharmaceutical compositions described herein may be appropriately set or adjusted in accordance with the dosage form, the route of administration, the degree or stage of a target disease, and the like.

In some embodiments, the dose administered to a subject is sufficient to prevent cancer, delay its onset, or slow or stop its progression or prevent a relapse of a cancer, or contribute to the overall survival of the subject. One skilled in the art will recognize that dosage will depend upon a variety of factors including the strength of the particular compound employed, as well as the age, species, condition, and body weight of the subject. The size of the dose will also be determined by the route, timing, and frequency of administration as well as the existence, nature, and extent of any adverse side-effects that might accompany the administration of a particular compound and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting, e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the tumor vaccine or vector encoding a tumor vaccine provided herein may vary depending on the specific tumor vaccine or vector encoding a tumor vaccine administered, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and most preferably causing complete regression of the cancer.

Examples of cancers that may treated by methods described herein include, but are not limited to, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing' s sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, rhabdosarcoma, serocystic sarcoma, synovial sarcoma, telangiectaltic sarcoma, Hodgkin's Disease, Non-Hodgkin's Lymphoma, multiple myeloma, neuroblastoma, bladder cancer, breast cancer, ovarian cancer, lung cancer, colorectal cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, superficial spreading melanoma, plasmacytoma, colorectal cancer, rectal cancer.

In some embodiments, the methods and compositions provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing' s sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary tumors that can be treated using the methods and compositions described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

Particular categories of tumors that can be treated using methods and compositions described herein include lymphoproliferative disorders, breast cancer, ovarian cancer, prostate cancer, cervical cancer, endometrial cancer, bone cancer, liver cancer, stomach cancer, colon cancer, colorectal cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

Cancers treated in certain embodiments also include precancerous lesions, e.g., actinic keratosis (solar keratosis), moles (dysplastic nevi), acitinic chelitis (farmer's lip), cutaneous horns, Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

Of special interest is the treatment of melanoma, breast cancer, prostate cancer pancreatic cancer, glioblastoma, renal cell carcinoma and colorectal cancer.

### EXAMPLES

The invention now being generally described, it will be more readily understood by reference to the following examples, which are included merely for purposes of illustration of certain aspects and embodiments of the present invention, and are not intended to limit the invention. As such, it will be readily apparent that any of the disclosed beneficial substances and therapies can be substituted within the scope of the present disclosure.

### Example 1

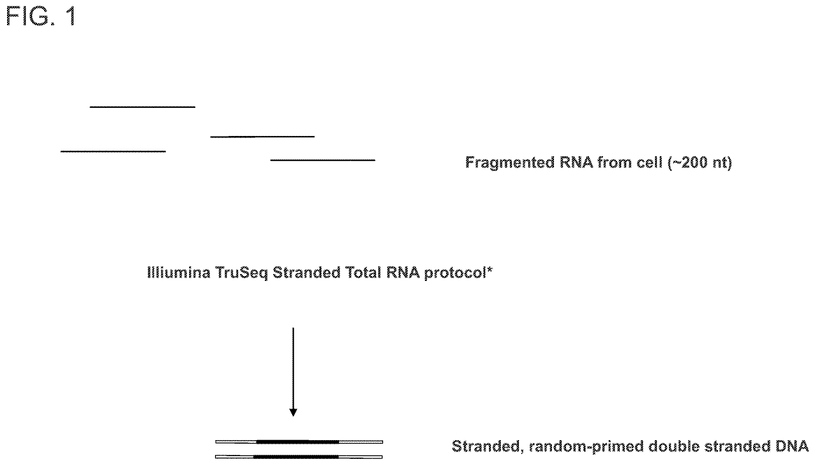

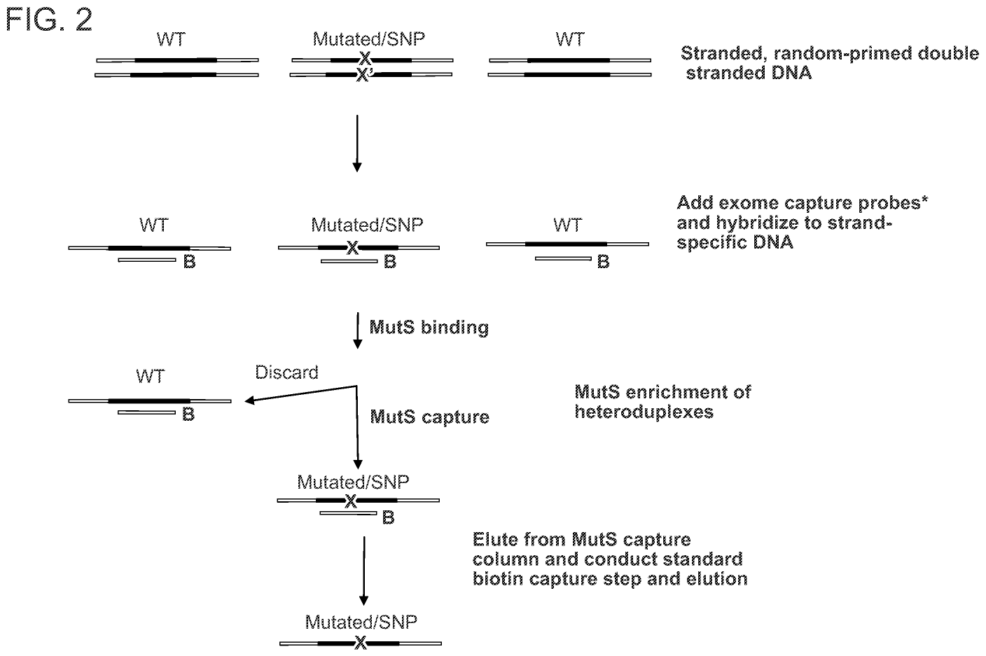

As illustrated in FIGS. 1 and 2, a collection of exome-selected, strand-specific-cDNA fragments is prepared from RNA from cells or Fresh Frozen Paraffin Embedded (FFPE) tissue slices from a patient that encode the entire or a selected fraction of proteins expressed by the cells. In some cases, the FFPE is enriched for regions that contain high concentrations of tumor cells by visual techniques or with magnification (Laser capture). If the cells are tumor cells, the library includes all or almost all neoantigens as well as tumor associated antigens and additional genomic regions near introns and in the untranslated regions which may contain neoantigen translation products not readily determinable by nucleic acid sequencing and use of an antigen prediction algorithm.

Optionally, as illustrated in FIGS. 2, the exome-captured fragments from tumor cells are enriched for mis-matched heteroduplex fragments first via incubation with protein MutS from bacterial species such as but not limited to E. coli or D. radionurans which is able to selectively bind to mis-matched double-stranded oligonucleotides and not perfectly matched duplexes, thus allowing a physical separation and enrichment of mis-matched double-stranded oligonucleotides and enrichment for mutation-containing fragments.

There are several ways to design the Exome Capture probes. The standard exome capture probes available are based on reference genome sequences and capture primarily coding region exons from all known CDS. This has three implications:(1) SNPs between the reference genome and the patient genome are consequently also enriched by MutS; (2) 5′ and 3′ UTRs are missed and there is limited length coverage of 5′ and 3′ exon-intron junctions; (3) because any given tumor histology typically expresses a more limited set of genes than the complete CDS, it is possible to design histology-specific capture probes. This can be based on sequence analysis of multiple “pure” tumor samples and inclusion of only those genes that are expressed above some baseline threshold. This can eliminate some aberrantly expressed genes in some patient's tumor. If a histology-specific capture set is used, some potential “stromal-associated” genes (e.g., Fibroblast activation protein (FAP) from cancer-associated fibroblasts that might contain useful epitope targets) may not be included. These may warrant separate inclusion.

Alternatively, to reduce the MutS enrichment of SNPs (the usual situation except for transplant applications), an exome capture probe set is designed based on the known location and frequency of SNPs. This probe set is designed around the locations of these SNPs so that SNP mis-match regions do not occur. The depth of SNP frequency which is designed around is analyzed. Capture probes that include 5′ and 3′ UTR and more extended intron regions are designed. A defined set of relevant stromal-associated target genes can be included in every histology-specific set.

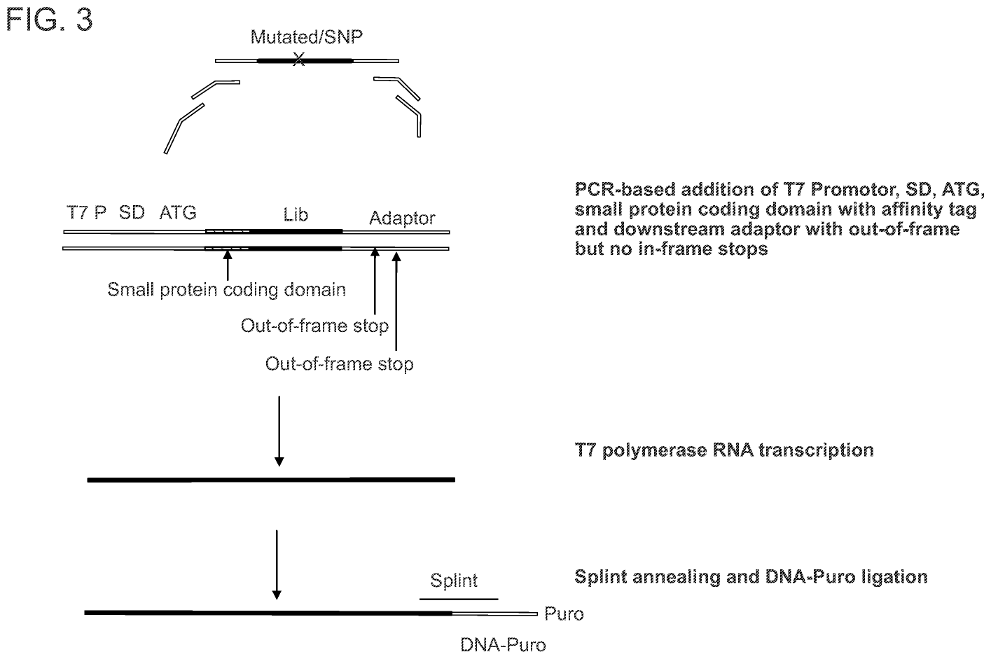

As illustrate in FIG. 3, the strand-specific fragments are inserted in the proper orientation by PCR or cloning between an upstream region containing a promotor for T7 RNA polymerase initiation followed by a translation initiation site (Shine-Dalgarno ribosome binding site or equivalent), an ATG (initiation) codon, and a coding sequence without a terminal stop codon for a small, soluble protein-coding domain (the small protein-coding domain contains translation stop sequences in both out-of-frame reading frames); and a downstream region containing a defined adapter sequence that can be used to enhance a later RNA/DNA ligation. The coding sequence without a terminal stop codon for a small, soluble protein-coding domain (the small protein-coding domain contains translation stop sequences in both out-of-frame reading frames) can also be at the downstream of the strand-specific fragments. In this alternative design, the 3′ end of the small protein-coding sequence can be used to enhance the later RNA/DNA ligation, and the adapter sequence is optional.

As illustrate in FIG. 3, the PCR/cloning product is used to produce RNA from the T7 initiation site. The RNA product is then ligated to a DNA oligonucleotide containing a puromycin molecule at its 3′ end (exemplary sequence dA21dCdC-Puromycin [5′ to 3′] (SEQ ID NO: 19)) in the presence of a splint DNA oligonucleotide which bridges the RNA and the puromycin-containing DNA oligonucleotide and then purified from excess linker and other reaction components

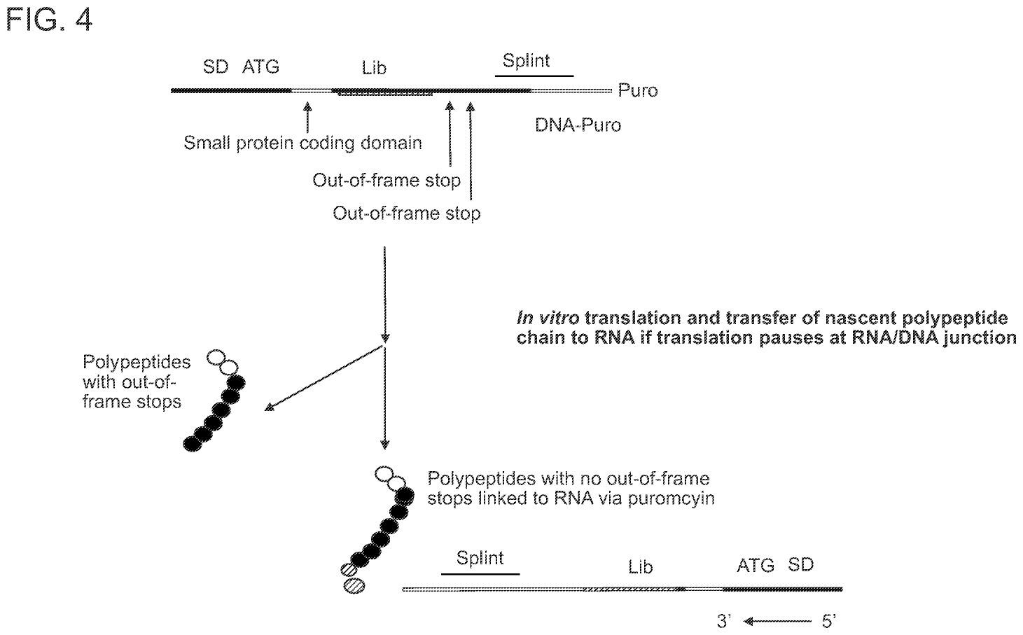

As illustrate in FIG. 4, the RNA is used in an in vitro translation reaction (rabbit reticulocyte lysate, wheat germ, E coli or equivalent) and any transcripts which read completely through the small protein domain and reach the ligated DNA oligonucleotide without encountering an out-of-frame translation stop codon will pause at the DNA sequence, allowing puromycin to enter the A site of the ribosome and link to the nascent polypeptide chain via normal peptidyl transferase activity, linking the successfully translated RNA to the polypeptide chain.

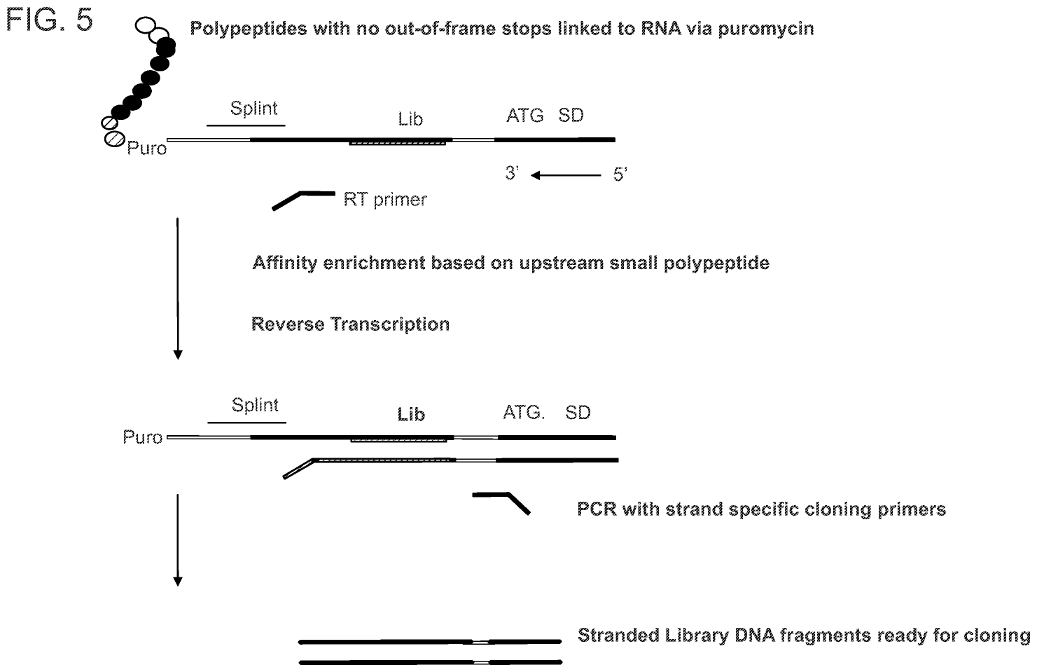

As illustrate in FIG. 5, any successfully linked mRNA/polypeptide chain molecules are enriched via binding to a column containing an affinity reagent for the small protein domain, producing a library of RNAs with in frame translation capability. If the RNA came from a tumor cell, the library can be used as a “whole tumor cell” vaccine which can be prepared from small, stored biopsy samples without the need to harvest significant quantities of fresh tissue, without the need for sequencing or bioinformatics and without the need for synthesis of multiple defined sequence oligonucleotides, and hence can be rapidly and inexpensively prepared. Reverse transcription and PCR are used-, in a strand-specific manner, to amplify the successful RNA inserts and the amplified product is cloned into a cloning vector to produce an RNA library containing the mini-proteome (AMPL-NA).

### Example 2

Total RNA is prepared from a human tumor cell line with whole exome sequencing data and a reasonably abundant and confirmed mutation burden. Illumina TruSeq RNA Exome library (stranded) is prepared. Exome capture is conducted. A sample of the library after exome sequencing is saved for pre-enriched sequencing.

A single-round of RNA display is conducted and PCR-amplified enriched library is obtained. Pre-enriched and enriched libraries are sequenced and analyzed.

Enrichment of fragments that enter and exit in the proper reading frame and support full-frame read-through is analyzed. Similar analyses focusing on known mutation-containing regions, or on SNPs identified by comparison of exome capture probe sequence and cell line sequence, are also conducted.

### Example 3

Total RNA is prepared from a human tumor cell line with whole exome sequencing data and a reasonably abundant and confirmed mutation burden. Illumina TruSeq RNA Exome library (stranded) is prepared. The RNA Exome library (stranded) is then hybridized to exome capture probes. Half (or another determined portion) of the sample is then processed for exome capture is used as un-enriched sample. For the other half or remaining portion of the sample is then bound to His-tagged MutS (ideally using D. radiodurans) and bound to nickel-coated ELISA plate. The library is then washed and digested with subtilisin, and processed for exome capture and mutation-enriched, exome-captured sample is PCR amplified. Pre-enriched and enriched libraries are sequenced and analyzed. Enrichment of sequences (# reads per total reads) containing known mutations is analyzed. Similar analysis focusing on SNPs identified by comparison of exome capture probe sequence and cell line sequence is also conducted.

### Example 4

Total RNA is prepared from a FFPE block for which standard whole exon sequencing (WES) is done in parallel. Illumina TruSeq RNA Exome library (stranded) is prepared. Exome capture is conducted. A sample of the library after exome sequencing is saved for pre-enriched sequencing. Single-round of RNA display is conducted and PCR-amplified enriched library is obtained. Pre-enriched and enriched libraries are sequenced and analyzed.

Enrichment of fragments that enter and exit in the proper reading frame and support full-frame read-through is analyzed. Similar analyses focusing on known mutation-containing regions, or on SNPs identified by comparison of exome capture probe sequence and cell line sequence, are also conducted.

### Example 5

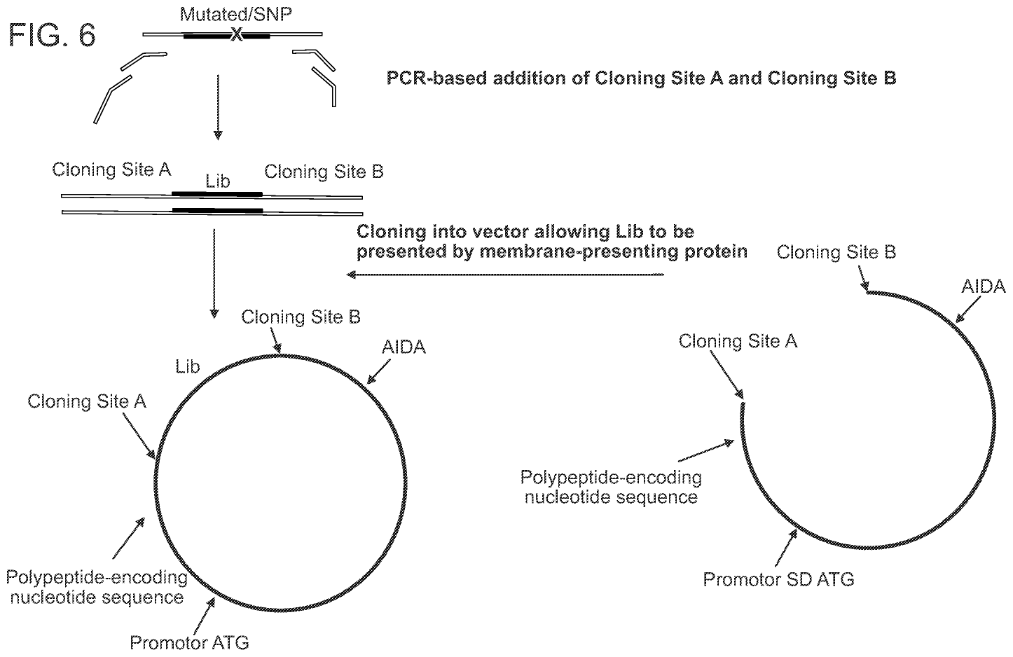

FIGS. 6, 87 and 8 presents an alternative approach to enrich the library of cDNA fragments for in-frame members. In these figures, the method of bacterial surface display is used to enrich in frame library members. Similar steps and gene constructs can be used to conduct phage display to enrich for in-frame library members, with specific modifications known to the person in the art, such as those described in e.g., U.S. Pat. Nos. 8,710,017, 8,685,893, and 8,372,954, all of which are incorporated by reference herein.

As illustrated in FIG. 6, the strand-specific fragments are extended by PCR to add oriented cloning sites. The cloning sites are then used to insert the library DNA fragments into a cloning vector so that the library DNA is positioned between a promotor, Shine-Dalgarno (SD) sequence, ATG initiation codon and polypeptide-encoding nucleotide sequence upstream of the library DNA and a membrane presenting protein-encoding sequence downstream of the library DNA (e.g., AIDA).

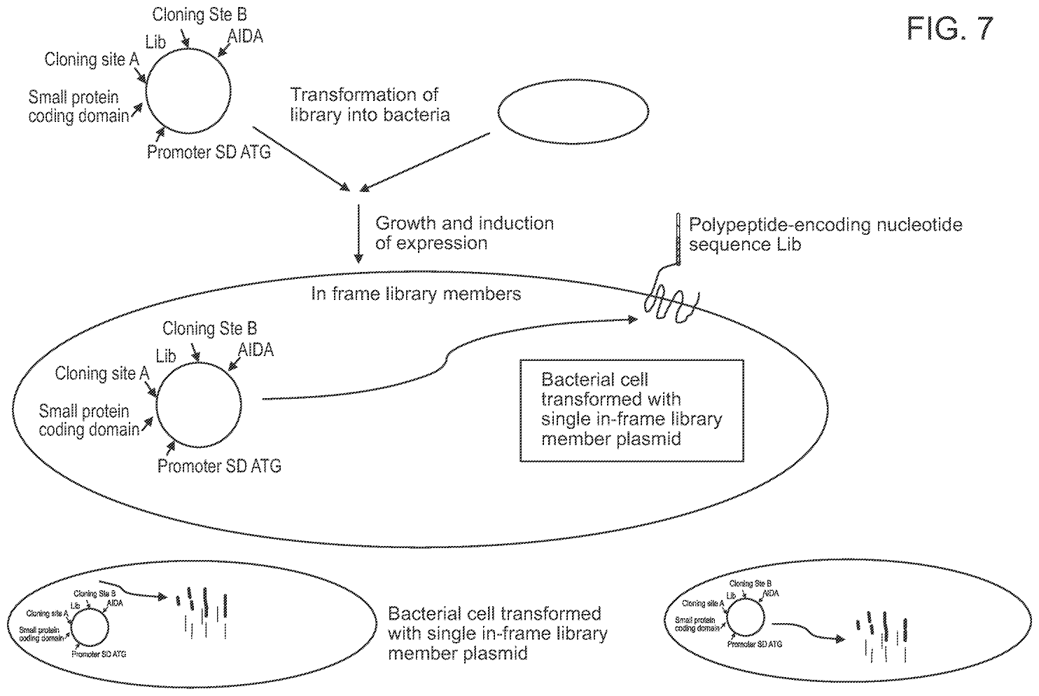

As illustrated in FIG. 7, the plasmids are transformed into a bacterial strain such as E. coli, and following growth and induction of expression by the promotor, the library is presented on the outer membrane of the bacteria if the library is in-frame with the polypeptide-encoding nucleotide sequence and the membrane presenting protein-encoding sequence. If translation initiates at the ATG and continues in frame to the end of the membrane presenting protein-encoding sequence (in-frame), the membrane presenting protein is inserted into the membrane and the polypeptide-encoding sequence is presented on the surface of the bacterium. If a stop codon is encountered prior to the membrane presenting protein is translated or the translation of the membrane presenting protein-encoding sequence is in the wrong reading frame, no membrane protein is produced and the membrane presenting protein is not presented on the surface of the cell.

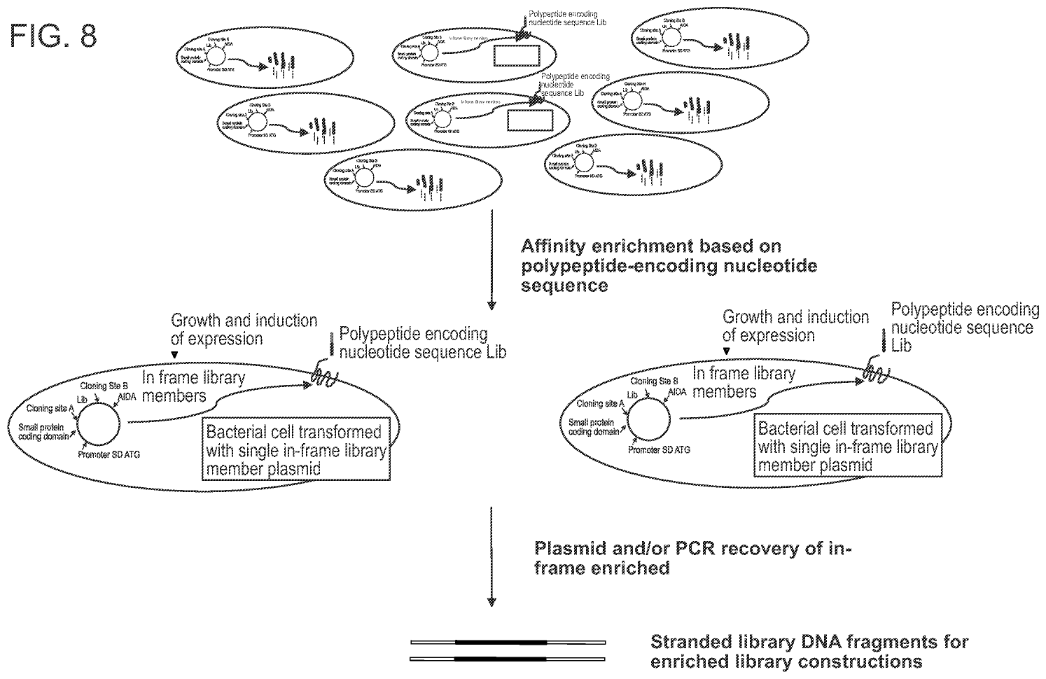

As illustrated in FIG. 8, the collection of bacteria containing in-frame and out-of-frame coding plasmids is exposed to an affinity reagent that binds to the polypeptide encoded by the polypeptide-encoding nucleotide sequence. Cells binding to the affinity reagent are separated from cells that contain plasmids that are out-of-frame and therefore do not bind to the affinity reagent. In some embodiments, magnetic beads can be attached to the affinity reagent allowing magnetic separation to separate cells bound to the affinity reagent from cells not bound to the affinity reagent. DNA is recovered from the cells that are bound to the affinity reagent, and the DNA is then PCR-amplified to prepare enriched stranded library fragments ready for enriched library construction.

### Example 6

### Preparation of Libraries

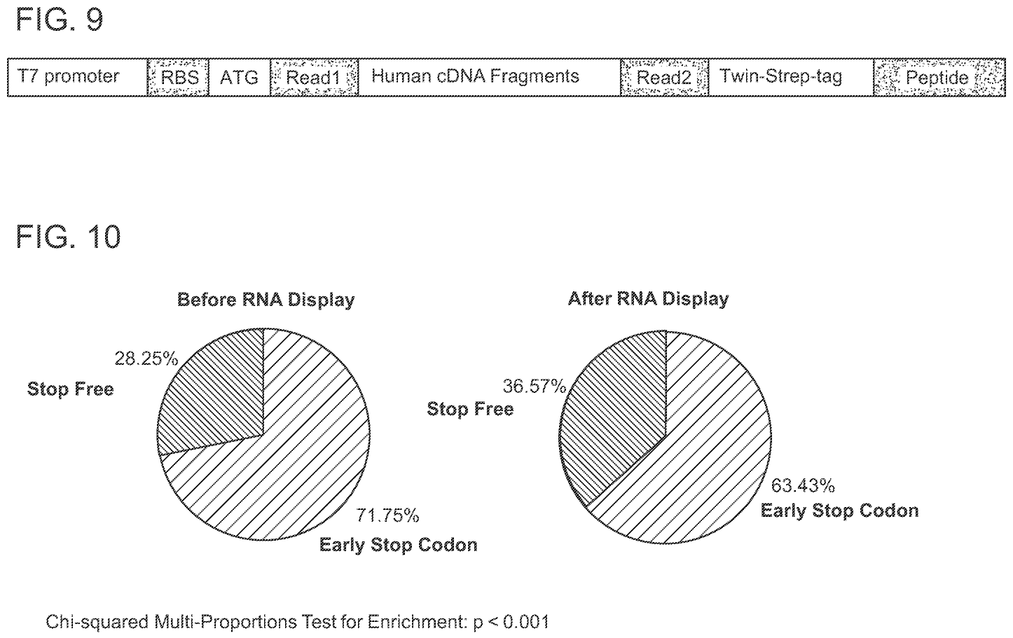

Total RNA was extracted from the melanoma cell line 13240-011 using the RNeasy Mini Kit (Qiagen 74104). After depleting ribosomal RNA using the NEBNext rRNA Depletion Kit v2 (New England BioLabs E7405), an RNA-seq library was prepared using the SEQuoia Complete Stranded RNA Library Prep Kit (Bio-Rad 17005726). The library was enriched for exome-containing fragments using the Twist Comprehensive Exome Kit (Twist Bioscience 102031) that employs baits based on the Consensus Coding Sequence (CCDS) database [Pujar et al., Nucleic Acids Res. 46(D1):D221-D228, 2018, doi: 10.1093/nar/gkx1031]. PCR with tailed primers was used to add 5′ and 3′ extensions to the library fragments in order to construct a library with the structure shown in FIG. 9.

### In Vitro Transcription

Using the transcription library as a template, RNA was synthesized using the HiScribe T7 High Yield RNA Synthesis Kit (New England BioLabs E2040S). Specifically, 8 μL 74 ng/μL Exome Capture Transcription Library was mixed with 2 μL10× Reaction Buffer, 2 μL 100 mM ATP, 2 μL 100 mM GTP, 2 μL 100 mM UTP, 2 μL 100 mM CTP, and 2 μL T7 RNA Polymerase Mix, then incubated at 37° C. for 2 hours. RNA was purified using the Monarch RNA Cleanup Kit (New England BioLabs T2030S) and recovered in 20 μL water. A 1 μL aliquot was used to make dilutions to analyze by Bioanalyzer (RNA 6000 Pico kit, Agilent 5067-1513) and the remainder was stored at −80° C.

### Ligation of RNA to Puromycin Oligo

Appending puromycin to the 3′ end of the in vitro transcribed RNA used the following two DNA oligos obtained from Integrated DNA Technologies (IDT): A27.C2.Puro (AAAAAAAAAAAAAAAAAAAAAAAAAAACC/3Puro/ (SEQ ID NO: 16)) and T10.ExPepSplint (TTTTTTTTTTCCAGTCGCTATAG (SEQ ID NO: 17)). The 13-nucleotide sequence at the 3′ end of T10.ExPepSplint is complementary to the 13-nucleotide sequence at the 3′ end of the in vitro transcribed RNA. Oligo A27.C2.Puro was phosphorylated by mixing 5 μL water, 2 μL 20 μM A27.C2.Puro, 1 μL 10× T4 Polynucleotide Kinase Reaction Buffer, 1 μL 10 mM ATP, and 1 μL 10 units/μL T4 Polynucleotide Kinase (New England BioLabs M0201S), incubating at 37° C. for 30 minutes, and transferring to ice. The following components were added: 7 μL water, 50 μL polyethylene glycol 8000 (from the T4 RNA Ligase 2 kit, New England BioLabs M0373L), 2 μL 20 μM T10.ExPepSplint, 4 μL 20 units/μL SUPERaseIn RNase inhibitor (Thermo Fisher, AM2696), and 8 μL 1.8 μg/μL in vitro transcribed RNA. After mixing well by pipetting up and down, the mixture was incubated at 65° C. for 2 minutes. While still warm, 10 μL 10× T4 RNA Ligase Reaction Buffer (from the T4 RNA Ligase 2 kit) was added, then the solution was mixed well by pipetting up and down, and placed on ice for 10 minutes. After incubation at room temperature for 5 minutes, 9 μL 25 units/μL SplintR Ligase (New England BioLabs M0375S) was added, then the solution was mixed well by pipetting up and down, and incubated at room temperature for 2 hours. The reaction was stopped by adding 2.5 μL 500 mM EDTA, pH 8.0 and mixing well. The RNA-puromycin product was purified using the NEBNext Poly(A) mRNA Magnetic Isolation Module (New England BioLabs E7490L) following the protocol provided with the module. For purification of the 100 μL ligation reaction, 100 μL well-resuspended NEBNext Magnetic Oligo d(T)25 Beads (SEQ ID NO: 18) were used. After following the protocol for binding to the beads and washing, the RNA-puromycin product was eluted with 20 μL nuclease-free water and transferred to a fresh tube. A 1 μL aliquot was used to make dilutions to analyze by Bioanalyzer (RNA 6000 Pico kit, Agilent 5067-1513) and the remainder was stored at −80° C. The yield of ligated product was 51.4%.

### RNA Display

In vitro translation was performed using components of the PURExpress In Vitro Protein Synthesis Kit (New England BioLabs E6800L). The reaction was assembled by mixing: 10 μL Solution A, 7.5 μL Solution B, 1 μL 20 units/μL SUPERase⋅In RNase inhibitor (Thermo Fisher, AM2696), and 6.5 μL 375 ng/μL RNA-puromycin product. Following incubation at 37° C. for 30 minutes, 3.1 μL 1 M MgCl₂ and 34.4 μL 1 M KCl were added to promote covalent linkage between translated peptides and puromycin. The reaction was incubated at room temperature for 30 minutes, then at −20° C. overnight. Peptide-RNA fusion products were purified using the NEBNext Poly(A) mRNA Magnetic Isolation Module (New England BioLabs E7490L) following the protocol provided with the module. For purification of the 62.5-μL translation reaction, 40 μL well-resuspended NEBNext Magnetic Oligo d(T)25 Beads (SEQ ID NO: 18) were used. After following the protocol for binding to the beads and washing, the peptide-RNA products were eluted with 20 μL 1 mM dithiothreitol (Teknova D9750) and transferred to a fresh tube. cDNA was synthesized using the RNA portion of the peptide-RNA products as template using the DNA oligo ExPep RT primer (CCAGTCGCTATAGCTGGCGTA (SEQ ID NO: 1)) obtained from IDT and 5× RT Buffer (75 mM Tris-HCl, pH 8.4, 375 mM KCl, 50 mM M MgCl₂), 25% (v/v) glycerol). For the cDNA reaction, a Hybridization Mix was made by mixing 15.4 μL water, 2 μL 10% NP-40 (Thermo Fisher 28324), 1.6 μL 25 mM each dNTPs (Thermo Fisher FERR1121), and 1 μL 10 μM ExPep RT primer. A 10 μL aliquot of peptide-RNA fusion sample was mixed with 10 μL Hybridization Mix and incubated at 65° C. for 1 minute followed by hold at 4° C. An RT Mix was prepared by mixing 26.5 μL water, 20 μL 5× RT Buffer, 1 μL 1 M dithiothreitol (Teknova D9750), 2 μL 40 U/μL RNaseOUT RNase inhibitor (Thermo Fisher 10777019), and 0.5 μL 200 U/μL SuperScript II Reverse Transcriptase (Thermo Fisher 18064014). After mixing the 20 μL peptide-RNA/Hybridization Mix sample with 20 μL RT Mix, the reaction was incubated at 42° C. for 60 minutes, 85° C. for 5 minutes, followed by hold at 4° C. For specific selection of peptide-RNA-cDNA products, the Twin-Strep-tag in the peptide portion was immobilized using MagStrep “type 3” XT Beads (Strep-Tactin XT coated magnetic beads, IBA LifeSciences 24090002). After transferring 25 μL well-suspended beads to a tube and placing in a magnetic stand, the supernatant was discarded. The beads were washed two times by resuspending in 200 μL Wash Buffer (100 mM 1 M Tris-HCl, pH 8.0, 150 mM NaCl, 1 mM EDTA), placing in magnetic stand, and discarding the supernatant. The 40 μL reverse transcriptase reaction was added to the beads and incubated on ice for 30 minutes, periodically flicking the tube gently to resuspend the beads. The sample was placed in a magnetic stand and the supernatant was discarded. The beads were washed three times by resuspending in 100 μL Wash Buffer, placing in magnetic stand, and discarding the supernatant. The beads were resuspended in 20 μL water and kept on ice. For the selected peptide-RNA-cDNA products, a sequencing library was restored using rhPCR amplification [Dobosy et al., BMC Biotechnol. 11:80, 2011, doi: 10.1186/1472-6750-11-80]. This amplification used the following two oligos obtained from IDT: P5.IDT312.Rd1x.x1 primer (AATGATACGGCGACCACCGAGATCTACACCTGACACAACACTCTTTCCCTACrA CGACa/3SpC3/, rA is riboA (SEQ ID NO: 2)) and P7.1DT024.Rd2x.x1 primer (CAAGCAGAAGACGGCATACGAGATAAGCACTGGTGACTGGAGTTCAGArCGTG Ta/3SpC3/, rC is riboC (SEQ ID NO: 3)). The 3′ ends of these oligos prime DNA synthesis in the Readl and Read2 segments, respectively, from the cDNA found in the peptide-RNA-cDNA products (see FIG. 9). The primers append the P5 and P7 segments, respectively, required for Illumina sequencing on the PCR products. The amplification also used 20× rhPCR Buffer (300 mM Tris-HCl, pH 8.4, 500 mM KCl, 80 mM MgCl₂) and the RNase H2 Enzyme Kit containing RNase H2 Enzyme and RNase H2 Dilution Buffer (IDT 11-02-12-01). RNase H2 was diluted to 20 mU/μL by mixing 1 μL 2 units/μL RNase H2 Enzyme and 99 μL H2 Dilution Buffer, then kept on ice. A 10 μL aliquot of the MagStrep bead suspension containing peptide-RNA-cDNA products was mixed with 2.5 μL 6 μM each P5.IDT312.Rdlx.xl/P7.IDT024.Rd2x.x1 primers. After preparing rhPCR Mix by combining 59.1 μL water, 4 μL 20× rhPCR Buffer, 1.3 μL 25 mM each dNTPs (Thermo Fisher FERR1121), 2 μL 20mU/μL RNase H2, and 1.6 μL 5 units/μL Hot Start Taq DNA Polymerase (New England BioLabs M0495L), 37.5 μL rhPCR Mix was added to the bead suspension/primers sample. PCR amplification was performed using the thermal protocol: 95° C. 30 seconds; 18 cycles of (96° C. 20 seconds, 62° C. 1 minute, 72° C. 1 minute); 4° C. hold. The reaction tube was placed in a magnetic stand and the supernatant was transferred to a fresh tube. PCR products were purified using the ProNex Size-Selective Purification System (Promega NG2003). The 50 μL amplification reaction was mixed with 70 μL (1.4×) ProNex beads and processed following the ProNex protocol. The RNA Display sequencing library resulting from the PCR was eluted with 20 μL ProNex Elution Buffer. A 1 μL aliquot was analyzed by Bioanalyzer (High Sensitivity DNA Kit, Agilent 5067-4626) and the remainder was stored at −20° C. The library was sequenced on the Illumina MiSeq following the manufacturer's instruction and using the 300-cycle MiSeq Reagent Kit v2 (Illumina MS-102-2022). The sequencing parameters were readl 150 cycles, index1 8 cycles, index2 8 cycles, read2 150 cycles. The sequencing results were analyzed to detect if the library inserts had intact open reading frames (ORFs). Results comparing the detection of full-length inserts with intact ORFs in the target reading frame for the constructs after Exome capture (“Before RNA Display”) and following RNA Display (“After RNA Display”) are shown in FIG. 10.

The fraction of the library inserts without stop codons (“Stop Free”) increased from 28% before RNA Display to 37% after RNA display (p<0.001). These results demonstrate that RNA display can enrich for human cDNA fragments with open reading frames in an exome-captured RNASeq library prepared from a patient with cancer.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of enriching a library of in-frame coding region fragments from a population of RNA transcripts, the method comprising:
(a) joining a population of RNA transcripts to puromycin-tagged linker polynucleotides, wherein:
the RNA transcripts in the population of RNA transcripts each comprise, in 5′ to 3′ order:
(i) a translation initiation site followed by any multiple of 3 nucleotides not encoding a stop codon;
(ii) a RNA sequence transcribed from a cDNA fragment sequence from a library of cDNA sequences from a tumor;
(iii) a polypeptide-encoding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames; and
the puromycin-tagged linker polynucleotides each comprise 3′ puromycin molecule,
wherein the 3′ end of RNA transcripts are joined to the 5′ end of the puromycin-tagged linker polynucleotides to generate puromycin-tagged RNA transcripts;
(b) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide-encoding nucleotide sequence, the puromycin will covalently link the translated polypeptide to the puromycin-tagged RNA transcript to form a polypeptide-linked RNA complex; and
(c) separating the polypeptide-linked RNA complexes from the RNA transcripts that are not in such complexes, thereby enriching a library of in-frame coding region fragments from a population of RNA transcripts.

2. A method of enriching a library of in frame coding region fragments from a population of RNA transcripts, the method comprising:
(a) joining a population of RNA transcripts to puromycin-tagged linker polynucleotides, wherein:
the RNA transcripts in the population of RNA transcripts each comprise, in 5′ to 3′ order:
(i) a translation initiation site followed by any multiple of 3 nucleotides not encoding a stop codon;
(ii) a polypeptide-encoding nucleotide sequence which is a multiple of 3 nucleotides in length and encoded by the reading frame initiating at the first 5′ nucleotide of the nucleotide sequence and lacks an nn-frame stop codon in that reading frame;
(iii) a RNA sequence transcribed from a cDNA fragment sequence from a library of cDNA sequences from a tumor; and
(iv) an adapter sequence which is a multiple of 3 nucleotides in length, and lacks stop codons in the reading frame beginning at the first 5′ nucleotide of the adapter sequence but contains stop codons in the other two reading frames, the puromycin-tagged linker polynucleotides each comprise 3′ puromycin molecule,
wherein the 3′ end of RNA transcripts arc joined to the 5′ end of the puromycin-tagged linker polynucleotides to generate puromycin-tagged RNA transcripts;
(b) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide-encoding nucleotide sequence, the puromycin will covalently link the translated polypeptide to the puromycin-tagged RNA transcript to form a polypeptide-linked RNA complex; and
(c) separating the polypeptide-linked RNA complexes from the RNA transcripts that are not in such complexes, thereby enriching a library of in-frame coding region fragments from a population of RNA transcripts.

3. The method of claim I or claim 2, wherein the population of RNA transcripts is joined to the puromycin-tagged linker polynucleotides by:
(a) contacting the RNA transcripts with splint polynucleotides and the puromycin-tagged linker polynucleotides, wherein:
the splint polynucleotides each comprise, in 3′ to 5′ order:
(I) a sequence complementary to the 3′ end of the polypeptide-encoding nucleotide sequence; and
(II) a linker-target sequence,
the puromycin-tagged linker polynucleotides each comprise, in 5′ to 3′ order:
(1) a sequence complementary to the linker-target sequence; and
(2) a puromycin molecule, and
wherein the polypeptide-encoding nucleotide sequence of the RNA transcripts hybridize to the sequence complementary to the 3′ end of the polypeptide-encoding nucleotide sequence of the splint polynucleotides, and the sequence complementary to the linker-target sequence of the linker polynucleotides hybridize to linker-target sequence of the splint polynucleotides;
(b) performing a ligation reaction to ligate the 3′ end of the RNA transcripts to the end of the puromycin-tagged DNA linkers to generate puromycin-tagged RNA transcripts.

4. The method of claim 3, wherein:
(i) the splint-target sequence is a poly-dT sequence and the sequence complementary to the splint-target sequence is a poly-dA sequence; or
(ii) the splint-target sequence is a poly-dA sequence and the sequence complementary to the splint-target sequence is a poly-dT sequence.

5. The method of any one of claims 1-4, the polypeptide-linked RNA complexes are separated from the RNA transcripts that are not in such complexes by affinity purifying the polypeptide-linked RNA complexes using a reagent that binds to the polypeptide encoded by the polypeptide-encoding nucleotide sequence.

6. The method of claim 5, further comprising perform an RT-PCR amplification reaction on the purified polypeptide-linked RNA complexes to generate an amplification product comprising an amplified DNA copy of the cDNA fragment sequence.

7. The method of claim 6, further comprising inserting the amplification product into a cloning vector.

8. The method of any one of claims 1 to 7, further comprising the step of generating the library of RNA transcripts prior to step (a) by performing a transcription reaction on a library of RNA expression constructs, wherein each RNA expression construct comprises:
(i) a transcription promoter;
(ii) a translation initiation site followed by any multiple of 3 nucleotides not encoding a stop codon;
(iii) a cDNA fragment sequence from a library of cDNA fragment sequences; and
(iv) a polypeptide coding nucleotide sequence which is a multiple of 3 nucleotides in length and encoded by the reading frame initiating at the first 5 nucleotide of the nucleotide sequence and lacks an in-frame stop codon in that reading frame but contains stop codons in each of the other two reading frames.

9. The method of claim 8, wherein each RNA expression construct further comprises an adapter sequence which is a multiple of 3 nucleotides in length, and lacks stop codons in the reading frame beginning at the first 5′ nucleotide of the adapter sequence but contains stop codons in the other two reading frames.

10. The method of claim 8 or claim 9, wherein the library of cDNA fragment sequences is enriched for exome-containing cDNA fragments.

11. The method of any one of claims 8-10, wherein the library of cDNA fragment sequences is enriched for mismatch-containing cDNA fragment sequences.

12. The method of any one of claims 8-11, wherein the translation initiation site comprises a Shine-Dalgamo sequence.

13. A method of enriching a library of in-frame coding region fragments from a population of cellular RNA fragments from a tumor, the method comprising:
(a) performing strand-specific random primed nucleic acid amplification reaction on a population of cellular RNA fragments to generate a population of cDNA fragments;
(b) contacting the population of cDNA fragments with exome capture probes thereby enriching the population of cDNA fragments for exome-encoding cDNA fragments to generate a library of exome-enriched cDNA fragments;
(c) generating RNA expression constructs comprising, (i) a transcription promoter; (ii) a translation initiation site followed by any multiple of 3 nucleotides not encoding a stop codon; (iii) one of the exome-enriched cDNA fragments from the library of exome-enriched cDNA fragments; (v) a polypeptide-coding nucleotide sequence which is a multiple of 3 nucleotides in length and encoded by the reading frame initiating at the first 5′ nucleotide of the nucleotide sequence and lacks an in-frame stop codon in that reading frame but contains stop codons in the other two reading frames;
(d) performing a transcription reaction using the RNA expression constructs to generate a library of RNA transcripts each comprising, in 5′ to 3′ order:
(i) a translation initiation site followed by any multiple of 3 nucleotides not encoding a stop codon;
(ii) a RNA sequence transcribed from a eDNA fragment sequence of the library of exome-enriched cDNA fragments;
(iii) a polypeptide-coding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames;
(e) joining a population of RNA transcripts to puromycin-tagged linker polynucleotides, wherein puromycin-tagged linker polynucleotides each comprise 3′ puromycin molecule and the 3′ end of RNA transcripts are joined to the 5′ end of the puromycin-tagged linker polynucleotides to generate puromycin-tagged RNA transcripts;
(f) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex; and
(g) separating the polypeptide-linked RNA complexes from the RNA transcripts that are not in such complexes, thereby enriching a library of in-frame coding region fragments from a population of cellular RNA fragments.

14. A method of enriching a library of in frame coding region fragments from a population of cellular RNA fragments from a tumor, the method comprising:
(a) performing strand-specific random primed nucleic acid amplification reaction on a population of cellular RNA fragments to generate a population of cDNA fragments;
(b) contacting the population of eDNA fragments with exome capture probes thereby enriching the population of eDNA fragments for exome-encoding cDNA fragments to generate a library of exome-enriched cDNA fragments;
(c) generating RNA expression constructs comprising, (i) a transcription promoter; (ii) a translation initiation site followed by any multiple of 3 nucleotides not encoding a stop codon; (iii) a polypeptide-coding nucleotide sequence which is a multiple of 3 nucleotides in length and encoded by the reading frame initiating at the first 5′ nucleotide of the nucleotide sequence and lacks an in-frame stop codon in that reading frame; (iv) one of the exome-enriched cDNA fragments from the library of exorne-enriched cDNA fragments; and (v) an adapter sequence which is a multiple of 3 nucleotides in length, and contains no stop codons in the reading frame beginning at the first 5′ nucleotide of the adapter sequence and stop codons in each of the other reading frames;
(d) performing a transcription reaction using the RNA expression constructs to generate a library of RNA transcripts each comprising, in 5′ to 3′ order:
(i) a translation initiation site followed by any multiple of 3 nucleotides not encoding a stop codon;
(ii) a polypeptide coding nucleotide sequence which is a multiple of 3 nucleotides in length and encoded by the reading frame initiating at the first 5′ nucleotide of the nucleotide sequence and lacks an in-frame stop codon in that reading frame;
(iii) a RNA sequence transcribed from a cDNA fragment sequence of the library of exome-enriched cDNA fragments; and
(iv) an adapter sequence which is a multiple of 3 nucleotides in length, and contains no stop codons in the reading frame beginning at the first 5′ nucleotide of the adapter sequence and stop codons in each of the other reading frames,
(e) joining a population of RNA transcripts to puromycin-tagged linker polynucleotides, wherein puromycin-tagged linker polynucleotides each comprise 3′ puromycin molecule and the 3′ end of RNA transcripts are joined to the 5′ end of the puromycin-tagged linker polynucleotides to generate puromycin-tagged. RNA transcripts;
(f) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex; and
(g) separating the polypeptide-linked RNA complexes from the RNA transcripts that are not in such complexes, thereby enriching a library of in-frame coding region fragments from a population of cellular RNA fragments.

15. The method of claim 13 or 14, wherein the population of RNA transcripts is joined to the puromycin-tagged linker polynucleotides by:
(a) contacting the RNA transcripts with splint polynucleotides and the puromycin-tagged linker polynucleotides, wherein:
the splint polynucleotides each comprise, in 3′ to 5′ order:
(I) a sequence complementary to the 3′ end of the polypeptide-encoding nucleotide sequence; and
(II) a linker-target sequence, the puromycin-tagged linker polynucleotides each comprise, in 5′ to 3′ order:
(1) a sequence complementary to the linker-target sequence; and
(2) a puromycin molecule, and
wherein the polypeptide-encoding nucleotide sequence of the RNA transcripts hybridize to the sequence complementary to the 3′ end of the polypeptide-encoding nucleotide sequence of the splint polynucleotides, and the sequence complementary to the linker-target sequence of the linker polynucleotides hybridize to linker-target sequence of the splint polynucleotides;
(b) performing a ligation reaction to ligate the 3′ end of the RNA transcripts to the 5′ end of the puromycin-tagged DNA linkers to generate puromycin-tagged RNA transcripts.

16. The method of claim 15, wherein:
(i) the splint-target sequence is a poly-dT sequence and the sequence complementary to the splint-target sequence is a poly-dA sequence; or
(ii) the splint-target sequence is a poly-dA sequence and the sequence complementary to the splint-target sequence is a poly-dT sequence.

17. The method of any one of claims 13-16, wherein step (b) further comprises contacting the population of cDNA fragments with a MutS protein, thereby enriching the population of cDNA fragments for mismatch-containing cDNA fragments due to either mutations or to single nucleotide polymorphisms.

18. The method of any one of claims 13-16, wherein step (h) further comprises contacting the library of exonie-enriched cDNA fragments with a MutS protein, thereby enriching the library of exoine-enriched cDNA fragments for mismatch-containing cDNA fragments due to either mutations or to single nucleotide polymorphisms.

19. The method of any one of claims 13 to 18, further comprising the step of preparing the population of cellular RNA fragments from a sample.

20. The method of claim 19, wherein the sample is a tumor sample, a normal tissue sample, a diseased tissue sample, a fresh sample, a frozen sample, and/or a paraffin embedded (FFPE) sample.

21. The method of claim 20, wherein the sample is a paraffin embedded (FFPE) tissue or tumor sample.

22. The method of any one of claims 17 to 20, further comprising obtaining the sample from a subject.

23. The method of any one of claims 13 to 22, wherein the cellular RNA fragments in the population of cellular RNA fragments are of between 150 and 250 nt in length.

24. The method of claim 23, wherein the cellular RNA fragments in the population of cellular RNA fragments are of about 200 nt in length.

25. The method of any one of claims 13 to 24. wherein e translation initiation site comprises a Shine-Dalgamo sequence.

26. The method of any one of claims 1 to 25, wherein the polypeptide-linked RNA complexes are separated from the RNA transcripts that are not in such complexes by affinity purifying the polypeptide-linked RNA complexes using a reagent that binds to the polypeptide encoded by the polypeptide-encoding nucleotide sequence.

27. The method of claim 26, further comprising performing an RT-PCR amplification reaction on the library of purified polypeptide-linked RNA complexes to generate amplification products comprising the sequence of the cDNA fragments.

28. The method of claim 27, further comprising contacting the amplification products with a MutS protein, thereby enriching the amplification products for mismatch-containing cDNA fragments due to either mutations or to single nucleotide polymorphisms.

29. The method of claim 27 or 28, further comprising inserting the amplification product into a vector to generate vectors comprising the sequence of the cDNA fragments.

30. The method of claim 29, wherein the vectors are cloning vectors.

31. The method of claim 29, wherein the vectors are expression vectors.

32. The method of claim 29, wherein the vectors are vaccine-coding vectors.

33. The method of claim 32, further comprising inserting the vaccine-coding vectors into bacteria and incubating the bacteria under conditions such that they express the vaccine encoded by the vaccine-coding vector.

34. The method of claim 32, further comprising inserting the vaccine-coding vectors into yeast and incubating the yeast under conditions such that they express the vaccine encoded by the vaccine coding vector.

35. The method of claim 32, further comprising subjecting the vaccine-coding vectors to an in vitro translation reaction to generate the vaccine encoded by the vaccine-coding vector.

36. The method of claim 29, further comprising transfecting or transducing the vectors into mammalian cells and incubating the mammalian cells under conditions such that they express the vaccine encoded by the vector.

37. The method of claim 36, wherein the mammalian cells are human cells.

38. The method of claim 29, further comprising transfecting or transducing the vectors into human cells ex vivo and delivering the human cells to a subject.

39. The method of claim 38, wherein the human cells are primary T cells or antigen-presenting cells isolated from the same subject or a different subject.

40. The method of claim 29, further comprising delivering the vectors to a subject such that the subject expresses the vaccine encoded by the vector.

41. The method of any one of claims 38-40, wherein the subject is a human.

42. A library of purified polypeptide-linked RNA complexes generated according to the method of claim 26.

43. Amplification products generated according to the method of claim 27 or 28.

44. Vectors generated according to the method of claim 29.

45. The vectors of claim 44, wherein the vectors are cloning vectors.

46. The vectors according to claim 44, wherein the vectors are expression vectors.

47. The vectors according to claim 44, wherein the vectors are vaccine-coding vectors.

48. A pharmaceutical composition comprising an amplification product of claim 43 and a pharmaceutically acceptable earlier.

49. A pharmaceutical composition comprising a vector of any one of claims 44 to 47 and a pharmaceutically acceptable carrier.

50. A method of generating a tumor vaccine comprising:
(a) generating cellular RNA fragments from a tumor sample of a subject;
(b) performing strand-specific random primed nucleic acid amplification reaction on the RNA fragments to generate cDNA fragments;
(c) contacting the cDNA fragments with exome capture probes thereby enriching the cDNA fragments for exotne-encoding cDNA fragments to generate a library of exome-enriched cDNA fragments;
(d) generating RNA expression constructs comprising. (i) a transcription promoter; (ii) a translation initiation site followed by any multiple of 3 nucleotides not encoding a stop codon; (iii) one of the exome-enriched cDNA fragments from the library of exome-enriched cDNA fragments; (iv) a polypeptide coding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames;
(e) performing a transcription reaction using the RNA expression constructs to generate a library of RNA transcripts each comprising, in 5′ to 3′ order:
(i) a translation initiation site followed by any multiple of 3 nucleotides not encoding a stop codon;
(ii) a RNA sequence transcribed from a cDNA fragment sequence of the library of exome-enriched cDNA fragments;
(iii) a polypeptide-coding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames,
(f) joining the RNA transcripts to puromycin-tagged linker polynucleotides, wherein puromycin-tagged linker polynucleotides each comprise 3′ puromycin molecule and the 3′ end of RNA transcripts are joined to the 5′ end of the puromycin-tagged linker polynucleotides to generate puromycin-tagged RNA transcripts;
(g) performing an in vitro translation reaction on the puromycin-tagged RNA transcripts, wherein, for each puromycin-tagged RNA fragment_{;} if the RNA sequence transcribed from a cDNA fragment sequence in a puromycin-tagged RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex;
(h) affinity purifying the polypeptide-linked RNA complexes using a reagent that binds to the polypeptide encoded by the polypeptide coding nucleotide sequence to generate a library of purified polypeptide-linked RNA complexes;
(i) performing an amplification reaction on the library of purified polypeptide-linked RNA complexes to generate amplification products comprising the sequence of the cDNA fragments; and
(j) generating a tumor vaccine from one or more of the amplification products of step (i).

51. A method of generating a tumor vaccine comprising:
(a) generating cellular RNA fragments from a tumor sample of a subject;
(b) performing strand-specific random primed nucleic acid amplification reaction on the cellular RNA fragments to generate cDNA fragments;
(c) contacting the cDNA fragments with exome capture probes thereby enriching the cDNA fragments for exome-encoding cDNA fragments to generate a library of exome-enriched cDNA fragments;
(d) generating RNA expression constructs comprising, (i) a transcription promoter; (ii) a translation initiation site followed by any multiple of 3 nucleotides not encoding a stop codon; (iii) a polypeptide coding nucleotide sequence which is a multiple of 3 nucleotides in length and encoded by the reading frame initiating at the first 5′ nucleotide of the nucleotide sequence and lacks an in-frame stop codon in that reading frame; (iv) one of the exome-enriched cDNA fragments from the library of exome-enriched cDNA fragments; and (v) an adapter sequence which is a multiple of 3 nucleotides in length, and contains no stop codons in the reading frame beginning at the first 5′ nucleotide of the adapter sequence and stop codons in each of the other reading frames;
(e) performing a transcription reaction using the RNA expression constructs to generate a library of RNA transcripts each comprising, in 5′ to 3′ order:
(i) a translation initiation site followed by any multiple of 3 nucleotides not encoding a stop codon;
(ii) a polypeptide coding nucleotide sequence which is a multiple of 3 nucleotides in length and encoded by the reading frame initiating at the first 5′ nucleotide of the nucleotide sequence and lacks an in-frame stop codon in that reading frame;
(iii) a RNA sequence transcribed from a cDNA fragment sequence of the library of exome-enriched cDNA fragments; and
(iv) an adapter sequence which is a multiple of 3 nucleotides in length, and contains no stop codons in the reading frame beginning at the first 5′ nucleotide of the adapter sequence and stop codons in each of the other reading frames,
(f) joining the RNA transcripts to puromycin-tagged linker polynucleotides, wherein puromycin-tagged linker polynucleotides each comprise 3′ puromycin molecule and the 3′ end of RNA transcripts are joined to the 5′ end of the puromycin-tagged linker polynucleotides to generate puromycin-tagged RNA transcripts;
(g) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex;
(h) affinity purifying the polypeptide-linked RNA complexes using a reagent that binds to the polypeptide encoded by the polypeptide coding nucleotide sequence to generate a library of purified polypeptide-linked. RNA complexes;
(i) performing an amplification reaction on the library of purified polypeptide-linked RNA complexes to generate amplification products comprising the sequence of the cDNA fragments; and
(j) generating a tumor vaccine from one or more of the amplification products of step (i).

52. The method of claim 50 or 51. wherein the population of RNA transcripts is joined to the puromycin-tagged linker polynucleotides by:
(a) contacting the RNA transcripts with splint polynucleotides and the puromycin-tagged linker polynucleotides, wherein:
the splint polynucleotides each comprise, in 3′ to 5′ order:
(I) a sequence complementary to the 3′ end of the polypeptide-encoding nucleotide sequence; and
(II) a linker-target sequence, the puromycin-tagged linker polynucleotides each comprise, in 5′ to 3′ order:
(1) a sequence complementary to the linker-target sequence; and
(2) a puromycin molecule, and
wherein the polypeptide-encoding nucleotide sequence of the RNA transcripts hybridize to the sequence complementary to the 3′ end of the polypeptide-encoding nucleotide sequence of the splint polynucleotides, and the sequence complementary to the linker-target sequence of the linker polynucleotides hybridize to linker-target sequence of the splint polynucleotides;
(b) performing a ligation reaction to ligate the 3′ end of the RNA transcripts to the 5′ end of the puromycin-tagged DNA linkers to generate puromycin-tagged RNA transcripts.

53. The method of claim 52, wherein:
(i) the splint-target sequence is a poly-dT sequence and the sequence complementary to the splint-target sequence is a poly-dA sequence; or
(ii) the splint-target sequence is a poly-dA sequence and the sequence complementary to the splint-target sequence is a poly-dT sequence.

54. The method of any one of claims 50-53, wherein the tumor sample is a fresh sample, a frozen sample, and/or a paraffin embedded (FFPE) sample.

55. The method of claim 54, wherein the sample is a paraffin embedded (FFPE) tumor sample.

56. The method of any one of claims 50 to 55, further comprising obtaining the tumor sample from a subject.

57. The method of any one of claims 50 to 56, wherein the cellular RNA fragments are of between 150 and 250 nt in length.

58. The method of claim 57, wherein the cellular RNA fragments are of about 200 nt in length.

59. The method of any one of claims 50 to 58, wherein the translation initiation site comprises a Shine-Dalgamo sequence.

60. The method of any one of claims 50 to 59, further comprising inserting the amplification product into a vaccine-coding vector to generate vaccine-coding vectors comprising the sequence of the cDNA fragments prior to step (j).

61. The method of claim 60. wherein step (j) comprises inserting the vaccine-coding vectors into bacteria and incubating the bacteria under conditions such that they express the vaccine encoded by the vaccine-coding vector.

62. The method of claim 60, wherein step (j) comprises inserting the vaccine-coding vectors into yeast and incubating the yeast under conditions such that they^{,} express the vaccine encoded by the vaccine-coding vector,

63. The method of claim 60, wherein step (j) comprises subjecting the vaccine-coding vectors to an in vitro translation reaction to generate the vaccine encoded by the vaccine-coding vector.

64. The method of claim 60, wherein step (j) comprises transfecting or transducing the vaccine-coding vectors into mammalian cells and incubating the mammalian cells under conditions such that they express the vaccine encoded by the vaccine-coding vector.

65. The method of claim 64, wherein the mammalian cells are human cells.

66. The method of any one of claims 50 to 65, further comprising administering the tumor vaccine to a subject.

67. The method of claim 60, wherein step (j) comprises transfecting or transducing the vaccine-coding vectors into human cells and delivering the human cells to a subject.

68. The method of claim 67, wherein the human cells are antigen-presenting cells isolated from the same subject or a different subject.

69. The method of claim 60, wherein step (j) comprises delivering the vaccine-coding vectors to a subject such that the subject expresses the vaccine encoded by the vaccine-coding vector.

70. The method of any one of claims 66 -69, wherein the subject is a human.

71. A method of treating a tumor, comprising administering the tumor vaccine generated according to a method of any one of claims 50 to 65 to a subject in need thereof.

72. A method of identifying drug targets comprising transfecting or transducing vectors generated according to claim 29 to cells and identifying in-frame coding region fragments that lead to a selectable phenotype.

73. The method of claim 72, wherein the vectors are transfected or transduced to cells in vitro or in vivo.

74. The method of claim 72 or 73, wherein the in-frame coding region fragments are either enriched or depleted in the cells with the selectable phenotype.

75. The method of any one of claims 72-74, wherein the in-frame coding region fragments positively or negatively alter an intracellular pathway.

76. The method of any one of claims 72-75, wherein the cells are normal cells and the selectable phenotype is a disease phenotype.

77. A method of enriching a library of in-frame coding region fragments from a population of cellular RNA fragments, the method comprising:
(a) performing strand-specific random primed nucleic acid amplification reaction on a population of cellular RNA fragments to generate a population of cDNA fragments;
(b) contacting the population of cDNA fragments with exome capture probes thereby enriching the population of cDNA fragments for exome-encoding cDNA fragments to generate a library of exome-enriched cDNA fragments;
(c) contacting the library of exome-enriched cDNA fragments with a MutS protein, thereby enriching the library of exome-enriched cDNA fragments for mismatch-containing cDNA fragments due to either mutations or to single nucleotide polymorphisms;
(d) generating RNA expression constructs comprising, (i) a transcription promoter; (ii) a translation initiation site followed by any multiple of 3 nucleotides not encoding a stop codon; (iii) one of the exome-enriched cDNA fragments from the library of exome-enriched cDNA fragments; (v) a polypeptide-coding nucleotide sequence which is a multiple of 3 nucleotides in length and encoded by the reading frame initiating at the first 5′ nucleotide of the nucleotide sequence and lacks an in-frame stop codon in that reading frame but contains stop codons in the other two reading frames;
(e) performing a transcription reaction using the RNA expression constructs to generate a library of RNA transcripts each comprising, in 5″ to 3′ order:
(i) a translation initiation site followed by any multiple of 3 nucleotides not encoding a stop codon;
(ii) a RNA sequence transcribed from a cDNA fragment sequence of the library of exome-enriched cDNA fragments;
(iii) a polypeptide-coding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames,
(f) joining the RNA transcripts to puromycin-tagged linker polynucleotides, wherein puromycin-tagged linker polynucleotides each comprise 3′ puromycin molecule and the 3′ end of RNA transcripts are joined to the 5′ end of the puromycin-tagged linker polynucleotides to generate puromycin-tagged. RNA transcripts:
(g) performing an in vitro translation reaction on the puromycin-tagged RNA transcripts, wherein, for each puromycin-tagged RNA fragment, if the RNA sequence transcribed from a cDNA fragment sequence in a puromycin-tagged. RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex; and
(h) separating the polypeptide-linked RNA complexes from the RNA transcripts that are not in such complexes, thereby enriching a library of in-frame coding region fragments from a population of RNA transcripts.

78. A method of enriching a library of in frame coding region fragments from a population of cellular RNA fragments, the method comprising:
(a) performing strand-specific random primed nucleic acid amplification reaction on a population of cellular RNA fragments to generate a population of cDNA fragments;
(b) contacting the population of cDNA fragments with exome capture probes thereby enriching the population of cDNA fragments for exome-encoding cDNA fragments to generate a library of exome-enriched cDNA fragments;
(c) contacting the library of exome-enriched cDNA fragments with a MutS protein, thereby enriching the library of exome-enriched cDNA fragments for mismatch-containing cDNA fragments due to either mutations or to single nucleotide polymorphisms;
(d) generating RNA expression constructs comprising, (i) a transcription promoter; (ii) a translation initiation site followed by any multiple of 3 nucleotides not encoding a stop codon; (iii) a polypeptide-coding nucleotide sequence which is a multiple of 3 nucleotides in length and encoded by the reading frame initiating at the first 5′ nucleotide of the nucleotide sequence and lacks an in-frame stop codon in that reading frame; (iv) one of the exome-enriched cDNA fragments from the library of exome-enriched cDNA fragments; and (v) an adapter sequence which is a multiple of 3 nucleotides in length, and contains no stop codons in the reading frame beginning at the first 5′ nucleotide of the adapter sequence and stop codons in each of the other reading frames;
(e) performing, a transcription reaction using the RNA expression constructs to generate a library of RNA transcripts each comprising, in 5′ to 3′ order:
(i) a translation initiation site followed by any multiple of 3 nucleotides not encoding a stop codon;
(ii) a polypeptide coding nucleotide sequence which is a multiple of 3 nucleotides in length and encoded by the reading frame initiating at the first 5′ nucleotide of the nucleotide sequence and lacks an in-frame stop codon in that reading frame;
(iii) a RNA sequence transcribed from a cDNA fragment sequence of the library of exome-enriched cDNA fragments; and
(iv) an adapter sequence which is a multiple of 3 nucleotides in length, and contains no stop codons in the reading frame beginning at the first 5′ nucleotide of the adapter sequence and stop codons in each of the other reading frames,
(f) joining the RNA transcripts to puromycin-tagged linker polynucleotides, wherein puromycin-tagged linker polynucleotides each comprise 3′ puromycin molecule and the 3′ end of RNA transcripts are joined to the 5′ end of the puromycin-tagged linker polynucleotides to generate puromycin-tagged RNA transcripts;
(g) performing an in vitro translation reaction on the puromycin-tagged RNA transcripts, wherein, for each puromycin4agged RNA fragment_{;} if the RNA sequence transcribed from a cDNA fragment sequence in a puromycin-tagged RNA transcript is in-frame with the translation initiation site, has no stop codons within that reading frame, and is in frame with the polypeptide coding nucleotide sequence, the puromycin will covalently link the translated polypeptide to the puromycin-tagged RNA transcript to form a polypeptide-linked RNA complex; and
(h) separating the polypeptide-linked RNA complexes from the RNA transcripts that are not in such complexes, thereby enriching a library of in-frame coding region fragments from a population of RNA transcripts.

79. The method of claim 77 or 78, wherein the population of RNA transcripts is joined to the puromycin-tagged linker polynucleotides by:
(a) contacting the RNA transcripts with splint polynucleotides and the puromycin-tagged linker polynucleotides, wherein:
the splint polynucleotides each comprise, in 3′ to 5′ order:
(I) a sequence complementary to the 3′ end of the polypeptide-encoding nucleotide sequence; and
(II) a linker-target sequence,
the puromycin-tagged linker polynucleotides each comprise, in 5′ to 3′ order:
(1) a sequence complementary to the linker-target sequence; and
(2) a puromycin molecule, and
wherein the polypeptide-encoding nucleotide sequence of the RNA transcripts hybridize to the sequence complementary to the 3′ end of the polypeptide-encoding nucleotide sequence of the splint polynucleotides, and the sequence complementary to the linker-target sequence of the linker polynucleotides hybridize to linker-target sequence of the splint polynucleotides;
(b) performing a ligation reaction to ligate the 3′ end of the RNA transcripts to the 5′ end of the puromycin-tagged DNA linkers to generate puromycin-tagged RNA transcripts.

80. The method of claim 79, wherein:
(i) the splint-target sequence is a poly-dT sequence and the sequence complementary to the splint-target sequence is a poly-dA sequence; or
(ii) the splint-target sequence is a poly-dA sequence and the sequence complementary to the splint-target sequence is a poly-dT sequence.

81. A method of enriching a library of in-frame coding region fragments from a population of cellular RNA fragments, the method comprising:
(a) performing strand-specific random primed nucleic acid amplification reaction on a population of cellular RNA fragments to generate a population of cDNA fragments;
(b) inserting the population of cDNA fragments into cloning vectors to generate a library of DNA constructs, wherein each DNA construct comprises, in 5′ to 3′ order:
(i) a promoter;
(ii) a translation initiation site followed by any multiple of 3 nucleotides not encoding a stop codon;
(iii) a polypeptide-encoding nucleotide sequence which is a multiple of 3 nucleotides in length and encoded by the reading frame initiating at the first 5′ nucleotide of the nucleotide sequence and lacks an in-frame stop codon in that reading frame;
(iv) one cDNA fragment from the population of cDNA fragments; and
(v) a membrane-presenting protein-encoding sequence,
(c) transforming the library of DNA constructs into cells,
(d) incubating the cells under conditions such that they express the DNA constructs;
(e) affinity purifying the cells that express a complete fusion protein comprising the polypeptide encoded by the polypeptide-encoding nucleotide sequence, the polypeptide encoded by the cDNA fragment, and the membrane-presenting protein using a reagent that binds to the polypeptide encoded by the polypeptide-.encoding nucleotide sequence;
(f) recovering in-frame cDNA fragment sequences from the purified cells by PCR amplification, thereby enriching a library of in-frame coding region fragments from a population of cellular RNA fragments.

82. The method of claim 81, wherein the cells are bacteria.

83. The method of claim 81 or 82, wherein the expression of the DNA constructs in the cells is inducible.

84. The method of any one of claims 81 to 83, wherein the membrane-presenting protein-encoding sequence encodes AIDA.

85. The method of any one of claims 81 to 84, wherein the step (a) further comprises contacting the population of cDNA fragments with exome capture probes thereby enriching the population of cDNA fragments for exome-encoding cDNA fragments to generate a library of exome-enriched cDNA fragments.

86. The method of any one of claims 81 to 84, wherein the step (a) further comprises contacting the population of cDNA fragments with a MutS protein, thereby enriching the population of cDNA fragments for mismatch-containing cDNA fragments due to either mutations or to single nucleotide polymorphisms.

87. The method of claim 85, wherein the step (a) further comprises contacting the library of exome-encoding cDNA fragments with a MutS protein, thereby enriching the library of exome-encoding cDNA fragments for mismatch-containing cDNA fragments due to either mutations or to single nucleotide polymorphisms.

88. The method of any one of claims 81 to 85, further comprising contacting the amplification products with a MutS protein, thereby enriching the amplification products for mismatch-containing cDNA fragments due to either mutations or to single nucleotide polymorphisms.

89. The method of any one of claims 81 to 88, further comprising the step of preparing the population of cellular RNA fragments from a sample.

90. The method of claim 89, wherein the sample is a tumor sample, a normal tissue sample, a diseased tissue sample, a fresh sample, a frozen sample, and/or a paraffin embedded (FFPE) sample.

91. The method of claim 90, wherein the sample is a paraffin embedded (FITE) tissue or tumor sample.

92. The method of any one of claims 89 to 91, further comprising obtaining the sample from a subject.

93. The method of any one of claims 81 to 92, wherein the cellular RNA fragments in the population of cellular RNA fragments are of between 150 and 250 nt in length.

94. The method of claim 93, wherein the cellular RNA fragments in the population of cellular RNA fragments are of about 200 nt in length.

95. The method of any one of claims 81 to 94, wherein the translation initiation site comprises a Shine-Dalgarno sequence.

96. The method of any one of claims 81 to 95, further comprising inserting the amplification product into a vector to generate vectors comprising the sequence of the cDNA fragments.

97. The method of claim 96, wherein the vectors are cloning vectors.

98. The method of claim 96, wherein the vectors are expression vectors.

99. The method of claim 96. wherein the vectors are vaccine-coding vectors.

100. The method of claim 99, further comprising inserting the vaccine-coding vectors into bacteria and incubating the bacteria under conditions such that they express the vaccine encoded by the vaccine-coding vector.

101. The method of claim 99, further comprising inserting the vaccine-coding vectors into yeast and incubating the yeast under conditions such that they express the vaccine encoded by the vaccine coding vector.

102. The method of claim 99, further comprising subjecting the vaccine-coding vectors to an in vitro translation reaction to generate the vaccine encoded by the vaccine-coding vector.

103. The method of claim 96, further comprising transfecting or transducing the vectors into mammalian cells and incubating the mammalian cells under conditions such that they express the vaccine encoded by the vector,

104. The method of claim 103, wherein the mammalian cells are human cells.

105. The method of claim 96, further comprising transfecting or transducing the vectors into human cells ex vivo and delivering the human cells to a subject.

106. The method of claim 105, wherein the human cells are primary T cells or antigen-presenting cells isolated from the same subject or a different subject.

107. The method of claim 96, further comprising delivering the vectors to a subject such that the subject expresses the vaccine encoded by the vector.

108. The method of any one of claims 105-107, wherein the subject is a human.

109. An amplification product generated according to the method of any one of claims 81-95.

110. Vectors generated according to the method of claim 96.

111. The vectors of claim 110, wherein the vectors are cloning vectors.

112. The vectors according to claim 110, wherein the vectors are expression vectors,

113. The vectors according to claim 110, wherein the vectors are vaccine-coding vectors.

114. A pharmaceutical composition comprising an amplification product of claim 109 and a phannaceutically acceptable carrier.

115. A pharmaceutical composition comprising a vector of any one of claims 110 to 113 and a pharmaceutically acceptable carrier.

116. A method of generating a tumor vaccine comprising:
(a) generating cellular RNA fragments from a tumor sample of a subject;
(b) performing strand-specific random primed nucleic acid amplification reaction on the RNA fragments to generate cDNA fragments;
(c) inserting the population of cDNA fragments into cloning vectors to generate a library of DNA constructs, wherein each DNA construct comprises, in 5′ to 3′ order:
(i) a promoter;
(ii) a translation initiation site followed by any multiple of 3 nucleotides not encoding a stop codon;
(iii) a polypeptide-encoding nucleotide sequence which is a multiple of 3 nucleotides in length and encoded by the reading frame initiating at the first 5′ nucleotide of the nucleotide sequence and lacks an in-frame stop codon in that reading frame;
(iv) one cDNA fragment from the population of cDNA fragments; and
(v) a membrane-presenting protein-encoding sequence,
(d) transforming the library of DNA constructs into cells,
(e) incubating the cells under conditions such that they express the DNA constructs;
(f) affinity purifying the cells that express a complete fusion protein comprising the polypeptide encoded by the polypeptide-encoding nucleotide sequence, the polypeptide encoded by the cDNA fragment, and the membrane-presenting protein using a reagent that binds to the polypeptide encoded by the polypeptide-encoding nucleotide sequence;
(g) recovering in-frame cDNA fragment sequences from the purified cells by PCR amplification,
(h) generating a tumor vaccine from one or more of the amplification products of step (g).

117. The method of claim 116, wherein the tumor sample is a fresh sample, a frozen sample, and/or a paraffin embedded (FFPE) sample.

118. The method of claim 117 wherein the sample is a paraffin embedded (FFPE) tumor sample.

119. The method of any one of claims 116 to 118, further comprising obtaining the tumor sample from a subject.

120. The method of any one of claims 116 to 119, wherein the cellular RNA fragments are of between 150 and 250 at in length.

121. The method of claim 120, wherein the cellular RNA fragments are of about 200 nt in length.

122. The method of any one of claims 116 to 121, wherein the translation initiation site comprises a Shine-Dalgamo sequence.

123. The method of any one of claims 116 to 122, further comprising inserting the amplification product into a vaccine-coding vector to generate vaccine-coding vectors comprising the sequence of the cDNA fragments prior to step (h).

124. The method of claim 123, wherein step (h) comprises inserting the vaccine-coding vectors into bacteria and incubating the bacteria under conditions such that they express the vaccine encoded by the vaccine-coding vector.

125. The method of claim 123, wherein step (h) comprises inserting the vaccine-coding vectors into yeast and incubating the yeast under conditions such that they express the vaccine encoded by the vaccine-coding vector.

126. The method of claim 123, wherein step (h) comprises subjecting the vaccine-coding vectors to an in vitro translation reaction to generate the vaccine encoded by the vaccine-coding vector.

127. The method of claim 123, wherein step (h) comprises transfecting or transducing the vaccine-coding vectors into mammalian cells and incubating the mammalian cells under conditions such that they express the vaccine encoded by the vaccine-coding vector.

128. The method of claim 127, wherein the mammalian cells are human cells.

129. The method of any one of claims 116 to 128, further comprising administering the tumor vaccine to a subject.

130. The method of claim 123, wherein step (h) comprises transfecting or transducing the vaccine-coding vectors into human cells and delivering the human cells to a subject,

131. The method of claim 130, wherein the human cells are antigen-presenting cells isolated from the same subject or a different subject.

132. The method of claim 123, wherein step (h) comprises delivering the vaccine-coding vectors to a subject such that the subject expresses the vaccine encoded by the vaccine-coding vector.

133. The method of any one of claims 129-132, wherein the subject is a human.

134. A method of treating a tumor, comprising administering the tumor vaccine generated according to a method of any one of claims 116 to 128 to a subject in need thereof.

135. A method of identifying drug targets comprising transfecting or transducing vectors generated according to claim 96 to cells and identifying in-frame coding region fragments that lead to a selectable phenotype.

136. The method of claim 135. wherein the vectors are transfected or transduced to cells in vitro or in vivo.

137. The method of claim 135 or 136, wherein the in-frame coding region fragments are either enriched or depleted in the cells with the selectable phenotype.

138. The method of any one of claims 135-137, wherein the in-frame coding region fragments positively or negatively alter an intracellular pathway.

139. The method of any one of claims 135-138, wherein the cells are normal cells and the selectable phenotype is a disease phenotype.

140. The method, library, amplification products, vectors, or pharmaceutical composition of any one of claims 1-139, wherein the polypeptide-encoding nucleotide sequence is at least 18 nucleotides in length.
